SEQUENCE LISTING (21 sequences)

SEQ ID NO: 1 - DNA, 1549 bp, Burkholderia glumae
acacatgcaagtcgaacggcagcacgggtgcttgcacctggtggcgagtggcgaacgggtgagtaatacatcggaacatgtcctgtagtgggggatagcccggcgaaagccggattaataccgcatacgatctacggatgaaagcgggggatctttggacctcgcgctatagggttggccgatggctgattagctagttggtggggtaaaggcctaccaaggcgacgatcagtagctggtctgagaggacgaccagccacactgggactgagacacggcccagactcctacgggaggcagcagtggggaattttggacaatgggcgaaagcctgatccagcaatgccgcgtgtgtgaagaaggccttcgggttgtaaagcacttttgtccggaaagaaatcctgagggctaatatccttcggggatgacggtaccggaagaataagcaccggctaactacgtgccagcagccgcggtaatacgtagggtgcgagcgttaatcggaattactgggcgtaaagcgtgcgcaggcggtttgttaagaccgatgtgaaatccccgggctcaacctgggaactgcattggtgactggcaagctagagtatggcagaggggggtagaattccacgtgtagcagtgaaatgcgtagagatgtggaggaataccgatggcgaaggcagccccctgggccaatactgacgctcatgcacgaaagcgtggggagcaaacaggattagataccctggtagtccacgccctaaacgatgtcaactagttgttggggattcatttccttagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatggtcggaatcccgaagagatttgggagtgctcgaaagagaaccgatacacaggtgctgcatggctgtcgtcagctcgtgtcgtgagatgttgggttaagtcccgcaacgagcgcaacccttgtccttagttgctacgcaagagcactctaaggagactgccggtgacaaaccggaggaaggtggggatgacgtcaagtcctcatggcccttatgggtagggcttcacacgtcatacaatggtcggaacagagggtcgccaacccgcgagggggagctaatcccagaaaaccgatcgtagtccggattgcactctgcaactcgagtgcatgaagctggaatcgctagtaatcgcggatcagcatgccgcggtgaatacgttcccgggtcttgtacacaccgcccgtcacaccatgggagtgggttttaccagaagtggctagtctaaccgcaaggaggacggtcaccacggtaggattcatgactggggtgaagtcgtaacaaggtagccgtatcggaaggtgcggctggatcacctcctttctcgagctaataccgcatacattgagcgttcacgcttatcggctgttgatcaagacagactcaggg

SEQ ID NO: 2 - DNA, 20 bp, Artificial Sequence
  Primer
cggaacatgtcctgtagtgg

SEQ ID NO: 3 - DNA, 18 bp, Artificial Sequence
  Primer
tctcagtcccagtgtggc

SEQ ID NO: 4 - DNA, 40 bp, Artificial Sequence
  Primer
gcgaggtccaaagatcccccaaagccggattaataccgca

SEQ ID NO: 5 - DNA, 42 bp, Artificial Sequence
  Primer
atagggttggccgatggctgatgtcctctcagaccagctact

SEQ ID NO: 6 - DNA, 23 bp, Artificial Sequence
  Primer
taaaggcctaccaaggcgacgat

SEQ ID NO: 7 - DNA, 1199 bp, Ajellomyces dermatitidis
ggatcattaacgcgccgtggggggttggacctcctagaccggaggaaccccggccccctcacctggccacccttgtctatttttacctgttgcttcggcgggcctgcagcgatgctgccgggggagttttcactccccgggcttgtgcccgccgaggacaccgctagaacttctggtgaacgattgacatctgagaaaataactataatcagttaaaactttcaacaacggatctcttggttccgacatcgatgaagaacgcagcgaaatgcgataagtaatgtgaattgcagaattccgtgaatcatcgaatctttgaacgcacattgcgccctctggtattccggggggcatgcctgtccgagcgtcattgcaaccctcaagcgcggcttgtgtgttgggccttcgtccccccgtggacgtgcccgaaatgcagcggcggcgtcgtgttccggtgcccgagcgtatggggctttgtcacccgctctagaggcccggccggctccggccccatctcaaacccttcgagggagggcggtcttcgggccggtctccccaccaggttgacctcggatcaggtaggaatacccgctgaacttaagcatatcaataagcggaggaaaagaaaccaacagggattgcctcagtaacggcgagtgaagcggcaaaagctcaaatttgaaatctggctcctttggggcccgagttgtaatttgcagaggatgcttcgggcgtggccgcggtctaagtcccctggaacggggcgtcgcagagggtgagaatcccgtcttcggccggccggcttcgcccatgtgaagctccttcgacgagtcgagttgtttgggaatgcagctctaaatgggtggtaaatttcatctaaagctaaatattggtcggagaccgatagcgcacaagtagagtgatcgaaagatgaaaagcactttgaaaagagagttaaacagcatgtgaaattgttgaaagggaagcgcttgcgaccagagtcggccgtgggggttcagcgggcattcgttgcccgtgcactcccccacgggcgggccagcgtcggtttcgacggccggtcaaaggcccccggaatgtgtcgcctctcggggcgtcttatagccgggggtgcaatgcggccagtcgggaccgaggaacgcgcttcggcacggacgctggcttaatggtcgtaagcgacc

SEQ ID NO: 8 - DNA, 17 bp, Artificial Sequence
  Primer
tccccaccaggttgacc

SEQ ID NO: 9 - DNA, 18 bp, Artificial Sequence
  Primer
tccaggggacttagaccg

SEQ ID NO: 10 - DNA, 44 bp, Artificial Sequence
  Primer
gaggcaatccctgttggtttcttcggatcaggtaggaatacccg

SEQ ID NO: 11 - DNA, 41 bp, Artificial Sequence
  Primer
cggcgagtgaagcggcaaaagccgaagcatcctctgcaaat

SEQ ID NO: 12 - DNA, 25 bp, Artificial Sequence
  Primer
cctccgcttattgatatgcttaagt

SEQ ID NO: 13 - DNA, 25 bp, Artificial Sequence
  Primer
aaatttgaaatctggctcctttggg

SEQ ID NO: 14 - DNA, 558 bp, Human papillomavirus type 18
tatataaaaaagggagtaaccgaaaacggtcgggaccgaaaacggtgtatataaaagatgtgagaaacacaccacaataccatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccgcatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgattccacaacatagctgggcactatagaggccagtgccattcgtgctgcaaccgagcacgacaggaaagactccaacgacgcagagaaacacaagtataa

SEQ ID NO: 15 - RNA, 376 nt, Human papillomavirus type 18
uauauaaaaaagggaguaaccgaaaacggucgggaccgaaaacgguguauauaaaagaugugagaaacacaccacaauaccauggcgcgcuuugaggauccaacacggcgacccuacaagcuaccugaucugugcacggaacugaacacuucacugcaagacauagaaauaaccuguguauauugcaagacaguauuggaacuuacagaggugccugcggugccagaaaccguugaauccagcagaaaaacuuagacaccuuaaugaaaaacgacgauuccacaacauagcugggcacuauagaggccagugccauucgugcugcaaccgagcacgacaggaaagacuccaacgacgcagagaaacacaaguauaa

SEQ ID NO: 16 - DNA, 16 bp, Artificial Sequence
  Primer
tgagaaacacaccaca

SEQ ID NO: 17 - DNA, 16 bp, Artificial Sequence
  Primer
cccagctatgttgtgg

SEQ ID NO: 18 - DNA, 39 bp, Artificial Sequence
  Primer
tgttcagttccgtgcacagatcgcgctttgaggatccaa

SEQ ID NO: 19 - DNA, 41 bp, Artificial Sequence
  Primer
tggaacttacagaggtgcctgcgtgtctaagtttttctgct

SEQ ID NO: 20 - DNA, 19 bp, Artificial Sequence
  Primer
gtagcttgtagggtcgccg

SEQ ID NO: 21 - DNA, 21 bp, Artificial Sequence
  Primer
gtgccagaaaccgttgaatcc

(19) United States
(12) Patent Application Publication
(10) Pub. No.: US 2016/0348189 A1
(43) Pub. Date: Dec. 1, 2016

(54) MOLECULAR DETECTION OF RNA

(71) Applicant: LUCIGEN CORPORATION, Middleton, WI (US)

(72) Inventor: Thomas William Schoenfeld, Topsfield, MA (US)

(73) Assignee: LUCIGEN CORPORATION, Middleton, WI (US)

(21) Appl. No.: 15/166,497

(22) Filed: May 27, 2016

Related U.S. Application Data
(60) Provisional application No. 62/167,483, filed on May 28, 2015.

(51) Int. Cl. C12Q 1/70; C12Q 1/68

(52) U.S. Cl. CPC C12Q 1/708; C12Q 1/689; C12Q 1/6895; C12Q 2600/158

(57) ABSTRACT

Methods of detecting RNA, such as ribosomal RNA (rRNA), messenger RNA (mRNA), and others. The methods include heating a cell comprising RNA in a solution to release the RNA from the cell, reverse transcribing the RNA into DNA with an enzyme, amplifying the DNA with the same enzyme, and detecting the amplified DNA. The heating, reverse-transcribing, and amplifying in at least some of the methods are performed at substantially the same temperature and a substantially constant temperature without adding additional reagents during or between the steps. The methods can be used to detect the presence of one cell type as distinguished from another cell type within a sample or to determine levels of gene expression, each without the need for elaborate extraction protocols.

### FIELD OF THE INVENTION

The invention is directed to amplifying RNA for detection and identification of unicellular and multicellular species and gene expression states of cells. The invention provides methods of isothermally amplifying and detecting ribosomal RNA (rRNA) for specifically detecting pathogenic microorganisms and other life forms and amplifying and detecting messenger RNA (mRNA) for detecting gene expression, all of which overcome the need for elaborate extraction protocols.

### BACKGROUND

Administration of the most effective care for infectious disease, cancer, or metabolic disorders often depends on the speed and reliability of diagnosis. Response time is often hampered not only by the rate of sample preparation and the incubation period of the diagnostic test itself, but also by the unavailability of sophisticated equipment and trained staff found only in well-equipped, centralized laboratories and by the requisite transport of samples to the lab and relay of results back to the patient caregiver. Ideally, rapid diagnosis would entail tests that are easy to perform by minimally trained personnel in the absence of sophisticated equipment and that provide an unambiguous result within a single patient encounter. These requirements drive the need for faster, more sensitive, more specific, more facile diagnostic tests.

It is widely held within the diagnostic arts that methods based on detecting the nucleic acids of an infectious agent are among the fastest, most reliable tests available for diagnosis of infectious disease. Likewise, nucleic acid-based tests for gene expression provide advantages in speed to result, sensitivity, and specificity compared to alternative methods and are particularly applicable to early detection of disease states including, but not limited to, cancer and metabolic disorders.

The most widely used nucleic acid detection methods are variants of the polymerase chain reaction (PCR), which provide very specific detection of as little as a single copy of nucleic acid. To provide this sensitivity, PCR is used to generate billions of copies of target sequence that are readily detected by a number of methods, most commonly fluorescence due to dye binding or activation of Fluorescence Resonance Energy Transfer (FRET) probes. More recently, numerous nucleic acid amplification methods have been introduced that share in common with PCR the highly sensitive and specific copying of the target nucleic acid, but differ in that they use essentially isothermal conditions, i.e., a single temperature throughout the amplification. A leading method of isothermal detection is loop-mediated isothermal amplification (LAMP) developed by Eiken Chemical, Tokyo (Notomi et al. 2000, U.S. Pat. No. 6,410,278). Isothermal amplification has several advantages mostly relating to speed of detection, simplicity of instrumentation and the ease of performance and interpretation of the test.

The great majority of tests for detecting both cellular and viral pathogens use as a target a defined sequence within the DNA (or RNA in the case of certain viruses) genome of the infectious agent. This inherently limits the sensitivity of detection in perfect cases to a single cell level based on the one or two copies of genomic DNA/RNA present, respectively, in, for example, prokaryotic cells and diploid eukaryotic cells. In practice, detection limits are often orders of magnitude higher due to imperfect amplification efficiency inherent in varying degrees to all methods of nucleic acid amplification. Cells also contain RNA, which can serve as an alternative detection target if the amplification chemistry is modified to support its detection. Much of the RNA in the cell is in the form of messenger RNA (mRNA), transfer RNA (tRNA), short nuclear RNA (snRNA) and other forms that may only be intermittently expressed and, when expressed, are usually present at copy numbers per cell of between one and one hundred. In contrast, ribosomal RNAs (rRNAs) are constitutively expressed, i.e., present in all living cells largely independent of metabolic state or developmental stage, and are often found at copy numbers between 10,000 and 100,000 in bacterial, fungal, and other cells. This high abundance can be exploited to greatly enhance sensitive detection.

The rRNA is highly conserved within microbial species, but distinct when different species are compared. Consequently, the gDNA sequences encoding these rRNAs have been widely used to establish the identities of microbes including bacteria, fungi, protozoans, and others (Dark et al. 2009, Hofman et al. 2015, Lo et al. 2015. Frickmann et al. 2015, Steenkeste et al. 2009. Jenkins et al. 2012, Ravindran et al. 2015, Ptaszynska et al. 2014. Huy et al. 2012, Xu and Li et al. 2012). This widespread use has led to public databases that provide nucleic acid sequences of rRNA from a vast array of microbial life that facilitate rapid design of nucleic tests (Cole et al. 2014, Wang et al. 2007, Wang et al. 2013. Fish et al. 2013. Cole et al. 2011. Cole et al. 2009. Cole et al. 2007. Cole et al. 2005. Cole et al. 2003. Maidak et al. 2001. Maidak et al. 2000, Maidak et al. 1999, Maidak et al. 1997, Maidak et al. 1996, Maidak et al. 1994. Larsen et al. 1993, Olsen et al. 1992). rRNA is quite stable in the cell compared to most species of RNA. improving the likelihood of detection.

In addition to the rRNA genes encoded in the prokaryotic and eukaryotic genomes, rRNA genes are also encoded in the mitochondrial genomes of eukaryotes. These mitochondrial rRNA genes are expressed, and the expressed mitochondrial rRNA can also be detected by the methods of this invention.

Detection of mRNA is widely used to discern metabolic changes associated with oncogenic transformation leading to cancers and with other metabolic disorders. mRNA detection is typically performed using two enzyme amplification mixes that enable reverse transcription PCR (RT PCR). These methods are hampered by the same limitations inherent in detecting rRNA, including complexity of the protocols and long times to result.

An important impediment to detecting any RNA, including rRNA and mRNA, is that to amplify the target, the chemistry of the amplification must provide a means to produce a reverse complimentary DNA copy of the RNA prior to amplifying the DNA copy by a process referred to in the art as reverse transcription (RT). This has traditionally depended on multiple enzymes, including one for the reverse transcription step and at least one for the amplification step. Certain enzymes, e.g. Thermus thermophilus Polymerase I, can be induced by modifying the ion content of the amplification mix to provide both reverse transcription and amplification. However, the performance by several criteria is inferior to the two-enzyme methods and this approach is not widely used in the art. More common are methods that use a retroviral reverse transcriptase, e.g., Moloney Murine Leukemia Virus (MMLV) or Avian Myeloblastosis Virus (AMV) reverse transcriptase, and a DNA polymerase, e.g., Taq DNA polymerase. These methods are fundamentally two step methods. Some protocols have simplified the two steps to provide a process that functionally appears to be a one-step process, but these processes are necessarily a compromise between the conditions most suitable for the enzymes required for reverse transcription and amplification. Because the temperatures that promote reverse transcription and amplification are different, there are usually separate incubations at different temperatures during the detection of RNA targets. Furthermore, rRNA is highly structured compared to other forms of RNA, i.e., it forms a highly stable double-stranded structure. This double-stranded structure relaxes upon heating to greater than about 60° C. The RNA rapidly re-anneals if cooled, so suitable reverse transcription must occur at the higher temperature.

With two exceptions, there is no reported precedence for isothermal amplification methods to detect rRNA. The related methods of transcription mediated amplification (TMA) and nucleic acid sequence-based amplification (NASBA) have been used to detect rRNA and are incorporated into commercial diagnostic products (e.g., the Hologic Aptima CT/NG assay). However, use of these products requires separate steps for target capture, amplification, and detection by hybridization, each requiring fluid transfer or addition and incubation at different temperatures. This complexity makes such tests unsuitable for operation outside a well-equipped lab by a highly-trained practitioner.

Numerous isothermal amplification tests have been reported that target genomic copies of the rRNA genes found in the DNA of both prokaryotes and eukaryotes. These tests are limited in their sensitivity to the number of copies of rRNA genes in the genomes or mitochondria and confer no substantial sensitivity advantage over any other DNA target.

Ideally, a method suitable for rapid detection of RNA targets, including rRNA or mRNA, should seamlessly provide reverse transcription as well as DNA amplification without additional processing or incubation steps. Suitably, the performance of the detection should be at least equivalent to the two-enzyme methods in sensitivity, specificity, and time to result. More suitably, this method would be compatible with direct detection of cells without additional processing, minimizing the need for elaborate sample extraction protocols. Demonstration of the sensitivity advantage would be provided by the ability to directly detect cells at less than single-cell sensitivity. More suitably, the chemistry would be simple enough that it facilitates use when sophisticated equipment and instrumentation and highly trained personnel are unavailable. Methods that exhibit at least some of these characteristics are needed.

### SUMMARY OF THE INVENTION

Provided are improved methods of detecting cells and determining cell states by amplifying and detecting RNA, such as rRNA and mRNA. The rRNA and mRNA amplification is preferably carried out using isothermal amplification. The methods exploit the unique properties of certain thermostable DNA polymerases including thermostability, reverse transcriptase activity (RNA-dependent DNA polymerase activity). DNA-dependent DNA polymerase activity, and strand displacement activity. The methods can provide processes suitable for use with limited equipment by practitioners lacking extensive training.

One version of the invention is a method of detecting RNA. The method comprises, heating a cell comprising RNA in a solution to release the RNA from the cell, reverse transcribing the RNA into DNA with an enzyme at a reverse-transcription temperature, amplifying the DNA with the enzyme at an amplification temperature, and detecting the amplified DNA. The enzyme is thermostable at the reverse-transcription temperature, is thermostable at the amplification temperature, has reverse transcriptase activity, has DNA-dependent DNA polymerase activity, and has strand displacement activity. The reverse-transcription temperature and the amplification temperature are substantially the same.

In some versions, the reverse-transcription temperature and the amplification temperature are each within 15° C. of each other.

In some versions, the reverse-transcription temperature and the amplification temperature are each within a range of from 65° C. to 90° C.

In some versions, the method comprises maintaining a substantially constant temperature during the reverse transcribing, from the reverse transcribing to the amplifying, and during the amplifying. The substantially constant temperature may be within a range spanning 15° C.

In some versions, the heating comprises heating the cell to a temperature substantially the same as the reverse-transcription temperature and the amplification temperature.

In some versions, the method comprises maintaining a substantially constant temperature during the heating, from the heating to the reverse transcribing, during the reverse transcribing, from the reverse transcribing to the amplifying, and during the amplifying. The substantially constant temperature may be within a range spanning 15° C.

In some versions, the heating, the reverse transcribing, and the amplifying are performed without adding additional reagents during or between these steps.

In some versions, the heating, the reverse transcribing, the amplifying, and the detecting are performed without adding additional reagents during or between these steps.

In some versions, the heating, the reverse transcribing, the amplifying, and the detecting are performed in a single container at a substantially constant temperature without adding additional reagents during or between the steps. The substantially constant temperature may be within a range spanning 15° C.

In some versions. RNA is ribosomal RNA (rRNA).

In some versions, the RNA is messenger RNA (mRNA).

In some versions, the solution comprises a primer that is complimentary to a ribosomal RNA sequence that is conserved among cells of a first cell type and is not present in ribosomal RNA of cells of a second cell type.

In some versions, the amplifying comprises isothermal amplification, such as loop-mediated isothermal amplification.

In some versions, the solution comprises a bodily fluid.

In some versions, the method further comprises mixing a liquid with dried solution components to generate the solution, wherein the dried solution components comprise the enzyme. In some versions, the method further comprises mixing a liquid comprising the cell with dried solution components to generate the solution, wherein the dried solution components comprise the enzyme. The liquid may comprise a bodily fluid.

In some versions, the heating does not include enzymatic digestion or physical agitation of the cell, and the heating releases the RNA from the cell without enzymatic digestion or physical agitation of the cell.

The objects and advantages of the invention will appear more fully from the following detailed description of the preferred embodiment of the invention made in conjunction with the accompanying drawings.

### BRIEF DESCRIPTION OF THE DRAWINGS

FIG. 1. Sensitivity of detection of B. glumae by detection of rRNA using RT LAMP. Indicated dilutions of B. glumae cells were tested using RT LAMP. CFUs shown in the axis were determined by plate count. Positive control (pos) was extracted B. glumae gDNA. NTC indicates a non-template control reaction lacking added nucleic acid or cells. Datapoints are the averages of triplicate reactions.

FIG. 2. Specificity of the 16S RT LAMP B. glumae test. The B. glumae reaction was used to test cultures of E. coli. S. aureus. B. glumae and P. aeruginosa cells. Positive reactions were detected by agarose gel electrophoresis. Included is a negative reaction lacking added nucleic acid or cells.

FIG. 3. Diagram of the target sequence of the B. dermatitidis test within the rRNA complex.

FIG. 4. Comparison between PCR, RT PCR, and RT LAMP sensitivity in detecting RNA and DNA. Dilutions of extracted RNA and DNA samples from Blastomyces, as well as non-template controls (NTC), were quantified by standard PCR or RT PCR as indicated (Panel A). The same sample dilutions were tested by RT LAMP (Panel B). The conditions from left to right in the set of bars under each dilution are as listed in the key from top to bottom.

FIG. 5. Direct detection of cells without extraction using the rRNA targeted LAMP. Sample of approximately 10⁶ cfu of cultured B. dermatitidis cells was tested in the presence and absence of saliva. A non-template control (NTC) was included.

FIG. 6. Cell lysis. About 2,000 cfu of cultured Blastomyces cells were added to each reaction except the non-template control (NTC) and the positive control, the latter which contained extracted PCR product. Pretreatment of the cells by a bead beating protocol (Panel A) or enzymatic lysis at varying temperature and incubation time as indicated (Panels B. C, and D) was compared to no treatment.

FIG. 7. Detection of HPV mRNA. Total nucleic acid was extracted from HeLa cell cultures either not treated (Panel A) or treated (Panel B) with RNase. The extracts were diluted in log 10 series. The dilutions as shown were tested by LAMP directed at the E6/E7 mRNA. Also run was a no-template control (NTC).

### DETAILED DESCRIPTION OF THE INVENTION

Provided are methods of detecting cells at sub-single-cell sensitivity. These methods rely on the detection of nucleic acid from the cells and can be performed on RNA, such as ribosomal RNA (rRNA), directly isolated from proliferating cells without additional treatment or handling. The methods are facilitated by a combination of thermal stability, reverse transcription activity, DNA-dependent DNA polymerase activity, and strand displacement activity of certain enzymes.

Also provided are methods to detect change in the metabolic state of a cell by detecting increases or decreases in gene expression based on changes in mRNA levels. This information may be used to infer progression of diseases such as cancer or to infer pathologic alterations of the metabolic state of the cells, providing a clinician with actionable information to guide treatment.

Thermal stability, reverse transcription activity, DNA-dependent DNA polymerase activity, and strand displacement activity are properties or activities of enzymes that are well-understood in the art. Thermal stability is the ability to maintain one or more enzymatic activities at elevated temperatures. Reverse transcription activity, also known as RNA-dependent DNA polymerase activity, is the activity of synthesizing a DNA molecule from an RNA template. DNA-dependent DNA polymerase activity is the activity of synthesizing a DNA molecule from a DNA template. Strand displacement activity is the activity of displacing downstream DNA annealed to an RNA or DNA template.

Enzymes for use in the present invention are preferably thermostable with regard to reverse transcription and DNA-dependent DNA polymerase activities at temperatures of at least about 40° C., at least about 45° C., at least about 50° C., at least about 55° C. at least about 60° C., at least about 61° C., at least about 62° C., at least about 63° C., at least about 64° C., at least about 65° C., at least about 66° C. at least about 67° C. at least about 68° C., at least about 69° C. at least about 70° C. at least about 71° C., at least about 72° C., or more. Such thermostability is preferably exhibited over a time period of at least about 10 minutes, at least about 15 minutes, at least about 20 minutes, at least about 25, minutes, at least about 30 minutes, at least about 35 minutes, at least about 40 minutes or more. There is no upper temperature limit on the thermostability of the enzymes that can be used in the invention, but it is rare for an enzyme to be thermostable at temperatures much higher than 110° C.

Consistent with the strand displacement activity, the enzymes suitable for use in the present invention are preferably devoid of 5′-3′ exonuclease activity. This permits use in isothermal amplification techniques that displace a previously synthesized strand from a nucleic acid template, such as loop-mediated isothermal amplification (LAMP) and others.

Exemplary enzymes suitable for use in various versions of the present invention include the PYROPHAGE 3173 Exonuclease Minus (Exo-) DNA Polymerase from Lucigen Corporation (Madison Wis.), the OMNIAMP Polymerase from Lucigen Corporation, the Bst 2.0 DNA Polymerase from New England BioLabs Inc. (Ipswich, Mass.), and the GspSSD LF DNA Polymerase from OptiGene (West Sussex, UK), among others. Also suitable are the polymerases and sequence variants thereof described in US 201210083018 (U.S. application Ser. No. 13/313,783), which is incorporated herein by reference.

Using an enzyme with the properties described above, the methods of the invention comprise reverse transcribing RNA released from a cell into DNA and amplifying the DNA at substantially the same temperature. As used herein. “temperature” refers to a single temperature value or a range of temperature values. As used herein. “substantially the same” used in reference to temperatures refers to temperature values that are the same or within about 30° C. about 25° C., about 20° C. about 19° C., about 18° C. about 17° C., about 16° C. about 15° C., about 14° C., about 13° C., about 12° C., about 11° C., about 10° C., about 9° C. about 8° C., about 7° C., about 6° C., about 5° C., about 4° C. about 3° C. about 2° C. or about 1° C. of each other.

The reverse transcription and amplification are each preferably performed at a temperature of from about 40° C. to about 90° C. from about 50° C. to about 90° C., from about 55° C. to about 90° C. from about 60° C. to about 90° C. from about 61° C. to about 90° C., from about 62° C. to about 90° C., from about 63° C. to about 90° C., from about 64° C. to about 90° C. from about 65° C. to about 90° C., from about 66° C. to about 90° C., from about 67° C. to about 90° C., from about 68° C. to about 90° C., from about 69° C. to about 90° C. from about 70° C. to about 90° C., from about 40° C. to about 80° C., from about 50° C. to about 80° C., from about 55° C. to about 80° C. from about 60° C. to about 80° C. from about 61° C. to about 80° C. from about 62° C. to about 80° C. from about 63° C. to about 80° C., from about 64° C. to about 80° C. from about 65° C. to about 80° C., from about 66° C. to about 80° C. from about 67° C. to about 80° C., from about 68° C. to about 80° C., from about 69° C. to about 80° C., or from about 70° C. to about 80°. In some versions of the invention, the reverse transcription and amplification are each preferably performed at a temperature up to about 75° C., up to about 76° C., up to about 77° C., up to about 78° C. up to about 79° C. up to about 80° C., up to about 81° C., up to about 82° C., up to about 83° C., up to about 84° C. up to about 85° C. up to about 86° C., up to about 87° C., up to about 88° C., up to about 89° C., or up to about 90° C.

The reverse transcription and amplification are each performed for a period of time. The period of time is preferably a time period of from about 2 minutes to about 120 minutes, such as about 2 minutes, about 10 minutes, about 15 minutes, about 30 minutes, about 45 minutes, about 60 minutes, about 75 minutes, about 90 minutes, about 105 minutes, about 120 minutes, or any ranges therebetween. Time periods above and below the stated values are also acceptable.

The reverse transcription and amplification may each comprise maintaining a substantially constant temperature for the period of time. “Substantially constant temperature” refers to a temperature that is maintained within a range spanning about 30° C. about 25° C., about 20° C. about 19° C., about 18° C. about 17° C., about 16° C. about 15° C., about 14° C. about 13° C., about 12° C., about 11° C. about 10° C. about 9° C., about 8° C. about 7° C., about 6° C. about 5° C., about 4° C., about 3° C. about 2° C., or about 1° C.

The RNA template used in the reverse transcription may be any type of RNA, including messenger RNA (mRNA), transfer RNA (tRNA), short nuclear RNA (snRNA) and ribosomal RNA (rRNA), among others. In one preferred version of the invention, rRNA is used as the template. In another preferred version, mRNA is the template.

The source of RNA for the reverse transcription is preferably a cell from which the RNA is released. The cell is preferably a whole or unlysed cell, or a population of whole or unlysed cells in a solution devoid of detectable amounts of free RNA (RNA not encompassed within a cell). The RNA is preferably released from the cell by heating the cell in a solution as described herein. The temperature to which the cell is heated is preferably substantially the same temperature at which the reverse transcription and/or the amplification is performed. Accordingly, the RNA is released from the cell by heating the cell to a temperature of from about 40° C. to about 90° C. from about 50° C. to about 90° C., from about 55° C. to about 90° C. from about 60° C. to about 90° C., from about 61° C. to about 90° C. from about 62° C. to about 90° C., from about 63° C. to about 90° C., from about 64° C. to about 90° C. from about 65° C. to about 90° C., from about 66° C. to about 90° C. from about 67° C. to about 90° C., from about 68° C. to about 90° C. from about 69° C. to about 90° C. from about 70° C. to about 90° C. from about 40° C. to about 80° C. from about 50° C. to about 80° C., from about 55° C. to about 80° C., from about 60° C. to about 80° C., from about 61° C. to about 80° C., from about 62° C. to about 80° C., from about 63° C. to about 80° C., from about 64° C. to about 80° C., from about 65° C. to about 80° C., from about 66° C. to about 80° C., from about 67° C. to about 80° C. from about 68° C. to about 80° C., from about 69° C. to about 80° C. or from about 70° C. to about 80°.

The heating is preferably performed for a period of time. The period of time is preferably a time period of from about 2 minutes to about 120 minutes, such as about 2 minutes, about 10 minutes, about 15 minutes, about 30 minutes, about 45 minutes, about 60 minutes, about 75 minutes, about 90 minutes, about 105 minutes, about 120 minutes, or any ranges therebetween. Time periods above and below the stated values are also acceptable. The heating may comprise maintaining a substantially constant temperature for the period of time.

In some versions, the RNA is released from the cell without enzymatic digestion or physical agitation of the cell. Examples of enzymatic digestion include digestion with one or more of Yatalase (Clontech, Mountain View. Calif.), Zymolase (Zymo Research. Irvine, Calif.) and CellLytic Y (Sigma-Aldrich). Examples of physical agitation include agitation with silica beads (bead beating), sonication, and shearing.

The release of RNA from the cell, the reverse transcription, and the amplification may be performed in the same solution. These steps may be carried out after adding the cell to the solution without addition, transfer, removal, purification, or extraction of components.

The solution may include a buffering agent. Any buffering agent suitable for buffering an aqueous solution at approximately neutral pH is acceptable. Suitable buffering agents are well known in the art. The solution is preferably buffered at a pH of from about 6 to about 9.

The solution may include a detergent. The detergent is preferably included in an amount of from about 0.01% w/v to about 2% w/v, such as about 0.01% w/v, about 0.025%, about 0.05% w/v, about 0.075% w/v, about 0.1% w/v, about 0.25%, about 0.5% w/v, about 0.75% w/v, about 1% w/v, about 1.25% w/v, about 1.5%, w/v, about 1.75% w/v, about 2% w/v, or any ranges therebetween. Amounts above and below these values are also acceptable. The detergent may be nonionic, ionic, or zwitterionic. Exemplary detergents include Triton X-100. Triton X-114, NP-40. Brij-35, Brij-58, Tween 20, Tween 80, octyl glucoside, octyl thioglucoside, sodium dodecyl sulfate (SDS), 3-[(3-cholamidopropyl)dimethylammonio]-1-propanesulfonate (CHAPS), and 3-[(3-cholamidopropyl)dimethylammonio]-2-hydroxy-1-propanesulfonate (CHAPSO).

The solution may also include a reducing agent. The reducing agent is preferably included in an amount of from about 0.1 μM to about 300 μM, such as about 0.1 μM, about 0.3 μM, about 1 μM, about 3 μM, about 10 μM, about 30 μM, about 100 μM, about 300, or any ranges therebetween. Amounts above and below these values are also acceptable. Exemplary reducing agents include tris(2-carboxyethyl)phosphine (TCEP) and dithiothreitol (DTT).

The solution may also include a magnesium salt. The magnesium salt is preferably included in an amount of from about 10 mM to about 100 mM, such as about 10 mM, about 20 mM, about 30 mM, about 40 mM, about 50 mM, about 60 mM, about 70 mM, about 80 mM, about 90 mM, about 100 mM, or any ranges therebetween. Amounts above and below these values are acceptable. Exemplary magnesium salts include magnesium sulfate and magnesium chloride.

The solution may also include an ammonium or potassium salt. Such salts are known to enhance DNA amplification reactions by facilitating nucleic acid strand separation. The ammonium or potassium salt may be included in an amount of from about 10 to about 100 mM, such as about 10 mM, about 20 mM, about 30 mM, about 40 mM, about 50 mM, about 60 mM, about 70 mM, about 80 mM, about 90 mM, about 100 mM, or any ranges therebetween. Amounts above and below these values are acceptable. Exemplary ammonium or potassium salts include ammonium sulfate and potassium chloride.

The solution may also include an enzyme as described above for performing the reverse transcription and DNA-dependent DNA polymerization. The enzyme is preferably present in an amount sufficient to generate a DNA copy of the RNA template and to amplify the DNA copy into a detectable amount.

The solution may also include deoxynucleotide triphosphates (dNTPs), such as deoxyadenosine triphosphate (dATP), deoxyguanosine triphosphate (dGTP), deoxythymidine triphosphate (dTTP), deoxyuridine triphosphate (dUTP), deoxycytidine triphosphate (dCTP), deoxyinosine triphosphate (dITP), and deoxyxanthosine triphosphate (dXTP). The deoxynucleotide triphosphates are preferably present in a combination and an amount sufficient to generate a DNA copy of the RNA template and to amplify the DNA copy into a detectable amount.

The solution may also include primers for the reverse transcription and DNA-dependent DNA polymerase reactions. The design of primers for reverse transcription and amplification, including isothermal amplification, is well-known in the art. The primers are preferably present in a combination and an amount sufficient to generate a DNA copy of the RNA template and to amplify the DNA copy into a detectable amount.

The solution may also comprise a DNA detection reagent. Exemplary DNA detection reagents include double-stranded DNA detection reagents and sequence-specific probes (hybridization probes). Double-stranded DNA detection reagents are well known in the art. Exemplary double-stranded DNA binding reagents include PICOGREEN (Life Technologies, Carlsbad, Calif.), SYBR Green (Life Technologies), ethidium bromide, and FIONAGREEN (Marker Gene Technologies, Inc., Eugene, Oreg.), among others. Exemplary sequence-specific probes include fluorophore-labeled probes and radiolabeled probes. Exemplary sequence-specific probes include SCORPIONS probes (Sigma-Aldrich, St. Louis, Mo.), molecular beacon probes, TAQMAN probes (Roche Molecular Diagnostics, Basel, Switzerland), Molecular Beacon probes, and LNA (Locked Nucleic Acid) probes, among others. The use of sequence-specific probes with the present invention may require DNA melting and annealing steps. Alternatively, the amplification product can be detected using a lateral flow detection device designed to provide a visual indication of the presence of a specific amplification product.

In some versions, the solution is devoid of one or more of DNA polymerases that have reverse transcriptase activity but not DNA-dependent DNA polymerase activity. DNA polymerases that have DNA-dependent DNA polymerase activity but not reverse transcriptase activity, and/or DNA polymerases having both reverse transcriptase activity and DNA-dependent DNA polymerase activity but only under different solution conditions, and nicking enzymes (strand-limited restriction endonucleases). In some versions, the solution is devoid of manganese, such that at least the reverse transcription and the amplification are performed in the absence of manganese.

The solution of the present invention may include any one or more of the above-mentioned components. For releasing RNA from a cell, it is preferred that the solution comprises one or more of the detergent, the reducing agent, the magnesium salt, the ammonium salt, and the potassium salt. Heating the cell in the presence of one or more of these components at the specified temperatures releases RNA from the cell in amounts sufficient to yield detectable amplified DNA, such as through partial or complete cell lysis. For reverse transcription and amplification, it is preferred that the solution comprises at least the enzyme, the deoxynucleotide triphosphates, and the primers. The other components mentioned above also help to facilitate the reverse transcription and amplification steps.

The solution preferably includes a solvent comprising water. The solvent may also include an organic solvent, such as dimethyl sulfoxide.

The solution in some versions includes a bodily fluid. The bodily fluid may be from the body of an animal or a plant. Exemplary types of suitable bodily fluids include intracellular fluids and extracellular fluids such as intravascular fluid (blood, plasma, serum), interstitial fluid, lymphatic fluid (sometimes included in interstitial fluid), transcellular fluid, and plant exudates. Exemplary suitable bodily fluids include amniotic fluid, aqueous humour, vitreous humour, bile, blood, breast milk, cerebrospinal fluid, cerumen, chyle, chime, endolymph, perilymph, plasma, exudates, feces (such as diarrhea), female ejaculate, gastric acid, gastric juice, lymph, mucus (including nasal drainage and phlegm), pericardial fluid, peritoneal fluid, pleural fluid, pus, rheum, saliva, sebum (skin oil), serous fluid, serum, semen, smegma, sputum, synovial fluid, sweat, tears, urine, vaginal secretion, and vomit, among others. The solution may comprise from 0.1% v/v to about 99% v/v of a bodily fluid, such as about 0.1% v/v, about 0.5% v/v, about 1% v/v, about 5% v/v, about 10% v/v, about 15% v/v, about 20% v/v, about 25% v/v, about 30% v/v, about 35% v/v, about 40% v/v, about 45% v/v, about 50% v/v, about 55% v/v, about 60% v/v, about 65% v/v, about 70% v/v, about 75% v/v, about 80% v/v, about 85% v/v, about 90% v/v, about 95% v/v, or any range therebetween.

In some versions of the invention, some or all of the solution components are initially provided together as a mix in dried form and are reconstituted with a liquid prior to use. The dried solution components may be reconstituted with a liquid comprising the cell from which the RNA is released. The reagents may be dried through lyophilization or other methods known in the art. The components in dried form are in a solid form, such as a powder, rather than in solution.

The dried liquid components may be reconstituted with a liquid comprising water. The liquid may further comprise an organic solvent, such as dimethyl sulfoxide. The liquid may also or alternatively comprise a bodily fluid, such as any of the bodily fluids listed above in any of the amounts listed above.

The DNA resulting from the reverse transcription may be amplified by any DNA amplification technique. Isothermal amplification is preferred. “Isothermal amplification” and grammatical variants thereof refer to amplification of DNA that occurs at substantially the same temperature. The temperature may vary over the course of the amplification procedure so long as the temperature remains substantially the same. In preferred versions of the invention, the isothermal amplification occurs in the absence of thermal cycling. Thermal cycling refers to the cycling between two or more defined temperatures for defined periods of time over the course of several cycles, such as 5, 6, 7, or more cycles, as occurs in PCR.

A number of isothermal amplification methods are known in the art. These include transcription mediated amplification (TMA), nucleic acid sequence-based amplification (NASBA), signal mediated amplification of RNA technology (SMART), strand displacement amplification (SDA), nicking enzyme amplification reaction (NEAR), rolling circle amplification (RCA), loop-mediated isothermal amplification of DNA (LAMP), isothermal multiple displacement amplification (MDA), helicase-dependent amplification (HDA), single primer isothermal amplification (SPIA), and cross primed amplification (CPA). Any of these isothermal amplification methods are potentially suitable for use in the present invention. Software and other methods for designing primers suitable for use in such isothermal amplification methods are well-known in the art. See, e.g., PrimerExplorer LAMP primer designing software from Eiken Chemical, Kimura et al. 2011, and others.

Preferred isothermal amplification methods are those that employ primers having a 5′ end that do not bind to template DNA when the 3′ is bound, having a portion complementary to downstream synthesized DNA, have a portion identical to downstream template DNA, and/or form loops by annealing to downstream synthesized DNA. Such primers are characteristic of such methods as LAMP and CPA, among others. See, e.g., Notomi et al. 2000; Xu and Hu et al. 2012; U.S. Pat. No. 6,410,278; U.S. Pat. No. 6,743,605; U.S. Pat. No. 6,764,821; U.S. Pat. No. 7,494,790; U.S. Pat. No. 7,468,245; U.S. Pat. No. 7,485,417; U.S. Pat. No. 7,713,691: U.S. Pat. No. 8,133,989: U.S. Pat. No. 8,206,902. U.S. Pat. No. 8,288,092: U.S. Pat. No. 8,445,664; U.S. Pat. No. 8,486,633; and U.S. Pat. No. 8,906,621.

The DNA that is amplified can be detected by detecting double-stranded DNA with double-stranded DNA detection reagents or specifically detecting amplified DNA sequences with sequence-specific probes. Any method of detecting amplified DNA is suitable for use in the present invention.

The methods of the invention can be used to specifically detect one type of cell as distinct from at least one other type of cell. This can be performed by targeting a distinguishing sequence of RNA. The distinguishing sequence is preferably conserved (i.e., identical or substantially identical) among cells of a cell type of interest and not present at a corresponding position or not present at all in the RNA of cells of at least one cell type that is not the cell type of interest. The targeting can occur by employing one or more primers complimentary to the distinguishing sequence in the amplification step and/or employing a probe complimentary to the distinguishing sequence in the detection step. The different types of cells may be from different domains, different kingdoms, different phyla, different classes, different orders, different families, different genera, different species, different subspecies, different variants, etc.

Accordingly, the solution in some versions of the invention comprises one or more primers that is (are) complimentary to an RNA sequence that is conserved among cells of a first cell type and is not present in nucleic acid, such as RNA, of cells of a second cell type or group of second cell types. The group of second cell types may comprise at least 2, at least 3, at least 4, at least 5, at least 10, at least 50, at least 100, at least 500, at least 1.000, at least 2.000, at least 5,000, or at least 10,000 cell types.

rRNA is replete with options for identifying distinguishing sequences for a large number of cells of interest. The rRNA sequences of a large variety of organisms are known and are well characterized. The sequences are maintained in public databases, and software for accessing such databases and designing sequences that can serve as distinguishing sequences are well known. See, for example, Cole et al. 2014. Wang et al. 2007. Wang et al. 2013, Fish et al. 2013, Cole et al. 2011, Cole et al. 2009. Cole et al. 2007, Cole et al. 2005, Cole et al. 2003, Maidak et al. 2001, Maidak et al. 2000, Maidak et al. 1999, Maidak et al. 1997, Maidak et al. 1996. Maidak et al. 1994, Larsen et al. 1993, Olsen et al. 1992, which describe the databases and software associated with the Ribosomal Database Project of Michigan State University.

The types of cells that can be detected include both eukaryotic cells and prokaryotic cells. Exemplary eukaryotic cells that can be detected include fungal cells, mammalian cells, protozoan cells and others. Exemplary prokaryotic cells that can be detected include bacterial cells and archaeal cells. The methods can be used to detect a eukaryotic cell as distinct from a non-eukaryotic cell, a prokaryotic cell as distinct from a non-prokaryotic cell, a fungal cell as distinct from a non-fungal cell, a mammalian cell as distinct from a non-mammalian cell, a bacterial cell as distinct from a non-bacterial cell, an archaeal cell as distinct from a non-archaeal cell, a type of eukaryotic cell as distinct from another type of eukaryotic cell, a type of prokaryotic cell as distinct from another type of prokaryotic cell, a type of fungal cell as distinct from another type of fungal cell, a type of mammalian cell as distinct from another type of mammalian cell, a type of bacterial cell as distinct from another type of bacterial cell, a type of archaeal cell as distinct from another type of archaeal cell, etc.

The RNA release, reverse transcription, amplification, and detection steps described herein are preferably performed individually or together at a substantially constant temperature. In some versions, the RNA release and reverse transcription steps are performed at a substantially constant temperature. In some versions, the reverse transcription and amplification steps are performed at a substantially constant temperature. In some versions, the amplification and detection steps are performed at a substantially constant temperature. In some versions, the RNA release, reverse transcription, amplification, and detection steps are all performed at a substantially constant temperature. In some versions, a substantially constant temperature is maintained during the RNA release step, from the RNA release step to the reverse transcription step, and during the reverse transcription step. In some versions, a substantially constant temperature is maintained during the reverse transcription step, from the reverse transcription step to and the amplification step, and during the amplification step. In some versions, a substantially constant temperature is maintained during the amplification step, from the amplification step to the detection step, and during the detection step. In some versions, a substantially constant temperature is maintained during the RNA release step, from the RNA release step to the reverse transcription step, during the reverse transcription step, from the reverse transcription step to the amplification step, during the amplification step, from the amplification step to the detection step, and during the detection step.

The RNA release, reverse transcription, amplification, and detection steps may together be performed for a period of time. The period of time is preferably a time period of from about 2 minutes to about 120 minutes, such as about 2 minutes, about 10 minutes, about 15 minutes, about 30 minutes, about 45 minutes, about 60 minutes, about 75 minutes, about 90 minutes, about 105 minutes, about 120 minutes, or any ranges therebetween. Time periods above and below the stated values are also acceptable. In some versions, the amplified DNA can be detected in the detecting step in a period of time less than about 120 minutes, less than about 105 minutes, less than about 90 minutes, less than about 75 minutes, less than about 60 minutes, less than about 45 minutes, less than about 30 minutes, less than about 25 minutes from the beginning of the heating step.

The methods of the invention permit detecting a cell with the above-mentioned steps in a single container at a substantially constant temperature without adding additional reagents during or between the steps. As the steps are not separated in space or distinctly in time, at least some of the steps of the methods may occur simultaneously. For example, some RNA molecules may be in the process of being reverse transcribed while others are still being released from the cell. Some DNA copies of the reverse transcribed RNA may be in the process of being amplified while some RNA molecules are being reverse transcribed and/or other RNA molecules are still being released from the cell. Some amplified DNA copies may begin to be detected while some DNA copies of the reverse transcribed RNA are in the process of being amplified, some RNA molecules are being reverse transcribed, and/or other RNA molecules are still being released from the cell. Accordingly, in some versions, the RNA release and reverse transcription steps at least partially overlap in time. In some versions, the reverse transcription and amplification steps at least partially overlap in time. In some versions, the amplification and detection steps at least partially overlap in time.

The methods described herein are capable of detecting cells present in the solution in an amount less than about 10⁻¹ cfu (colony forming units), such as about less than about 10⁻² cfu or less than about 10⁻³ cfu. Sensitivity in detecting cells present in the solution in an amount as low as 10^{-3.5}, 10⁻⁴, 10⁻⁵, or lower is predicted.

The methods described herein are capable of detecting RNA present in the solution in a copy number less than about 1.000 copies, less than about 500 copies, less than about 250 copies, less than about 200 copies, less than about 150 copies, less than about 100 copies, less than about 75 copies, or less than about 50 copies. Sensitivity in detecting RNA present in the solution in an amount as low as 40 copies, 35 copies, 30 copies, 25 copies, 20 copies, 15 copies, 10 copies, 5 copies, or lower is predicted.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### Example 1

### Detection of Bacterial Ribosomal RNA

### Summary

In one embodiment, the invention comprises a method of detecting bacterial species at a high sensitivity by using RT LAMP to reverse transcribe thermally relaxed rRNA into DNA reverse complement that serves as a template for DNA amplification by the same enzyme. This process comprises a single manipulation and incubation at a single temperature to specifically detect the bacterial species of interest. In the example provided, the test targets are the V1 and V2 variable regions of the 16S rRNA. Bacterial rRNA comprises several forms commonly referred to as 5S, 16S, and 23S. These forms are present at essentially equimolar amounts and any variable region of any of these rRNA forms would suffice to provide a target for sensitive, specific detection. All known species of bacteria produce rRNA. Any bacterial species can be detected with modest alteration of the primer sequence and reaction conditions and without undue experimentation.

In the present example, 16S rRNA was directly detected from liquid bacterial cultures of Escherichia coli, Burkholderia glumae, Staphylococcus aureus and Elizabethkingia meningoseptica. Primers were designed to target regions of divergence from other strains. The region 1-300 was used for E. coli, B. glunmae and S. aureus. Region 300-600 was used for E. meningoseptica. These regions cover variable regions V1 and V2 of the 16S rRNA gene. The reaction buffer was used to resuspend, lyse, and extract the rRNA in a single step. The reaction was transferred immediately without further addition of fluid or other manipulation to a Bio-Rad cfx96 realtime PCR thermocyler set to isothermal incubation at 70° C. and used to perform LAMP amplification. Notwithstanding the use of a laboratory-based instrument in the example, it should be appreciated that the underlying amplification chemistry is suitable for detection of amplification by any of a number of means well-established in the art that also fulfill the need for ease of use and portability. These means include fluorescence detection in a portable format, nucleic acid lateral flow, colorimetric detection, turbidimetric detection and others.

### Design of LAMP Primers for Burkholderia

The sequence of the 16S rRNA gene (SEQ ID NO:1) of Burkholderia glunmae was used to design primers for LAMP. The 16S rRNA genes of Burkholderia glunmae and related species were compared using the Megalign® computer program (DNAStar, Madison Wis.) to identify regions conserved among target species and distinct relative to species likely to co-occur in the same samples. These regions of relative inclusivity and exclusivity were targeted during design of primers using PrimerExplorer (Eiken Chemical). The resulting primers (SEQ ID NOS:2-6) were synthesized by IDT (Coralville, Iowa).

### Testing of the Sensitivity of the Burkholderia LAMP

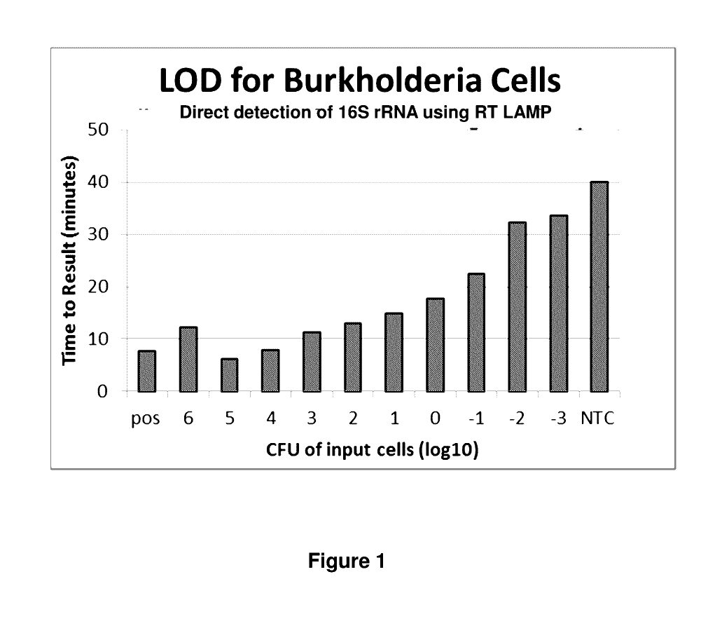

Burkholderia glumae cells were grown to late log phase at 30° C. in Luria Broth and serially-diluted over a range of 10 to 10⁻⁹ in Luria broth for determination of cell density and in TT buffer (10 mM 1.25 mM TCEP (tris(2-carboxyethyl)phosphine), 25 mM Tris-HCl, pH 8.0) for LAMP detection. Serial dilutions between 10⁻⁴ and 10⁻⁹ were plated on Yeast Tryptone agar to determine cell density, measured as colony forming units (cfu), as is commonly practiced in the art. The entire dilution series (10⁻⁰ to 10⁻⁹) was prepared in TT for use in LAMP reactions. For amplification, a mix comprising 0.2 μM primer B16F3 (SEQ ID NO:2), 0.2 μM primer B16B3 (SEQ ID NO:3), 1.6 μM primer B16FIP (SEQ ID NO:4), 1.6 μM primer B16BIP (SEQ ID NO:5), 0.4 μM primer B16_7 (SEQ ID NO:6), 20 mM K-MOPS pH 7.9, 10 mM NH₄SO₄, 0.8 mM dATP, 0.8 mM dCTP, 0.8 mM dGTP, 0.8 mM dTTP, 10 mM MgSO₄. 0.3% CHAPS, 2 mM Fiona Green dye. 0.72 U/μl PYROPHAGE 3173 exo-DNA Polymerase (Lucigen Corporation, Madison, Wis.) was used. 1.5 μl of each cell dilution was added to 23.5 μl of amplification mix and the reactions transferred immediately to a Bio-Rad realtime PCR thermocyler run isothermally at 72° C. for the duration of amplification. Fluorescence was monitored using the FAM/SYBR setting on the instrument at 30 second intervals. Time to result (TTR) was determined based on the time the relative fluorescence took to cross a theoretical threshold set at 10% of the maximum. The results are shown in FIG. 1. When the TTRs are graphed against the cell counts from the original log-phase culture, a detection sensitivity of at least 10⁻³ cfu was determined.

### Specificity of Detecting Bacterial Ribosomal RNA

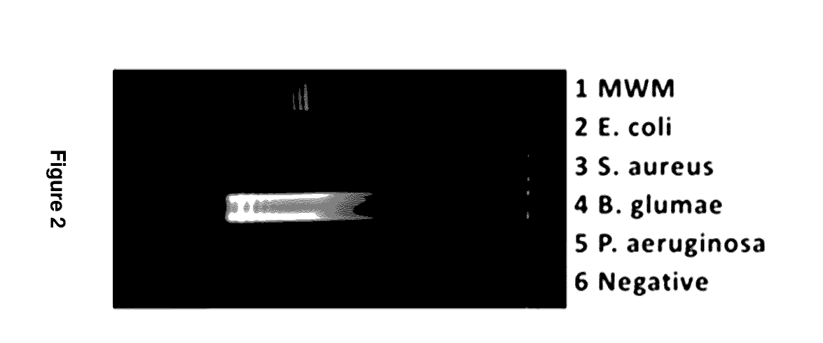

LAMP reactions against the non-target bacterial strains were used to assess specificity. The same reaction mix and incubation conditions as described above were used to test cell cultures (circa 10⁶ cells per reaction) of E. coli. S. aureus, B. glumue and E. meningoseptica. Results of LAMP amplification as detected by agarose gel are shown in FIG. 2. Tests were performed with other primer sets against target and non-target species with similar specificity for target (not shown). The primers were specific for the target species.

### Example 2

### Detection of Eukaryotic Ribosomal RNA

### Summary

In a further embodiment, the invention comprises a means of detecting a eukaryotic species by targeting the eukaryotic rRNA by direct reverse transcription and amplification. In the example shown, the D1 and D2 regions of the 28S rRNA are targeted for detection. All eukaryotes produce rRNA of different forms commonly referred to as 5S, 5.8S, 18S and 28S, and all forms are present in essentially equimolar amounts and contain regions of varying distinctness. A nucleic acid amplification test can successfully target any of these forms with similar advantages in sensitivity. Any eukaryotic pathogen can be detected, including fungus, e.g. Candida spp., protozoa, e.g. Plasmodium falciparum, parasitic worm. e.g. Ascaris lumbricoides, or any other eukaryotic infectious agent, by targeting one of these rRNA sequences. These tests can be developed without undue experimentation. In addition to the rRNA genes encoded in the nuclear genome, eukaryotes also encode prokaryotic-like rRNA genes in the genomes of subcellular organelles, e.g. mitochondria, chloroplasts, apicoplasts, that also would serve as potential targets for pathogen-specific detection. This reasoning extends to nonpathogenic bacterial and eukaryotic microbes or cells. The gDNA copy has been used as a target (Oriero et al. 2015, Haanshuus et al. 2013), but not the rRNA itself.

In the present example, the RT LAMP was used to detect a group of dimorphic fungi by targeting the rRNA. This group of fungi is characterized by two life forms, a multicellular mold state and a unicellular yeast state with the transition between the two life forms determined by temperature. The mold state predominates in the environment and the yeast state predominates after infection of exothermic hosts, including humans. For historical reasons, the mold state and yeast states of a single species are referred as Ajellomyces dennatitidis and Blastomyces dermatitidis, respectively. The respective mold and yeast states of a second distinct species are referred to as Ajellomyces capsulatum and Histoplasma capsulatum. In both cases, the latter term is used in the example although both forms can be equivalently detected. A third species of dimorphic fungus is called Coccidioides immitis in both the mold and the yeast form. The rRNA sequences of these three species were directly detected using the rRNA test.

RT LAMP was used in the examples. It should be appreciated that other isothermal and thermocycled amplification processes can be used, including SDA. HDA. NEAR, PCR and others to directly detect the rRNA and confer similar advantages in sensitivity, specificity and ease of use. Notwithstanding the use of RT LAMP in the examples, modification of the processes allows the practice of the invention using other amplification methods.

Design of LAMP Primers for B. dermatitidis

A target sequence of the rRNA complex in B. dermatitidis (SEQ ID NO:7) was used to design primers for LAMP. The rRNA complexes of B. dermatitidis strains and related species were compared using the Megalign® computer program (DNAStar, Madison Wis.) to identify regions conserved among target species and distinct relative to species likely to co-occur in the same samples. These regions of relative inclusivity and exclusivity were targeted during design of primers using PrimerExplorer (Eiken Chemical). The resulting primers (SEQ ID NOS:8-13) were synthesized by IDT (Coralville, Iowa).

### Direct Detection of Fungal Ribosomal RNA by LAMP

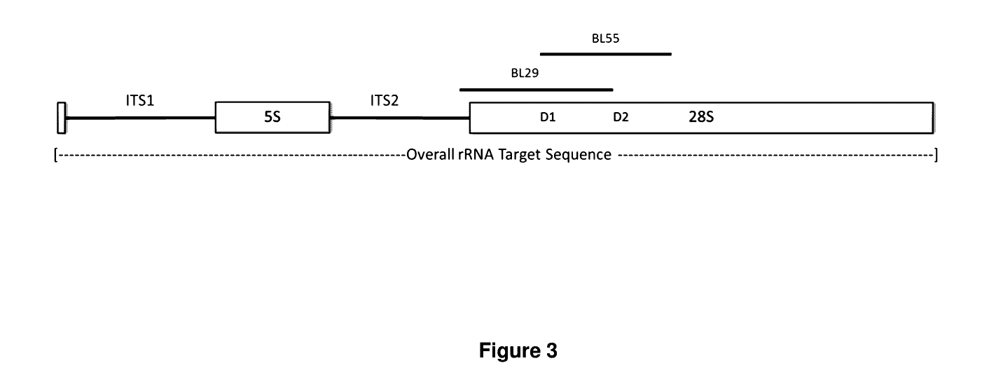

LAMP reactions targeting the 28S rRNA of the fungal pathogen Blastomyces dermatitidis were developed against the target sequence of SEQ ID NO:7. The locations of the primer sites are shown diagrammatically in FIG. 3. For amplification, a mix comprising 0.2 μM primer BL29F3 (SEQ ID NO:8), 0.2 μM primer BL29B3 (SEQ ID NO:9), 1.6 μM primer BL29FIP (SEQ ID NO:10), 1.6 μM primer BL29BIP (SEQ ID NO:11), 0.4 μM primer BL29LF1 (SEQ ID NO:12), 0.4 μM primer BL29LB1 (SEQ ID NO:13), 20 mM K-MOPS pH 7.9, 10 mM NH₄SO₄, 0.8 mM dATP, 0.8 mM dCTP, 0.8 mM dGTP, 0.8 mM dTTP, 10 mM MgSO₄. 0.3% CHAPS, 2 mM Fiona Green dye, 0.72 U/μl PYROPHAGE 3173 exo-DNA Polymerase (Lucigen Corporation, Madison, Wis.) was used. 2.0 μl of each cell dilution was added to 23.0 μl of amplification mix and the reactions transferred immediately to a Bio-Rad cfx96 realtime PCR thermocyler run isothermally at 68° C. for the duration of the test. Fluorescence was monitored using the FAM/SYBR setting on the instrument at 30 second intervals. Time to result (TTR) was determined based on the time for the relative fluorescence to cross a theoretical threshold set at 10% of the maximum.

Concordance of the RT LAMP with RT PCR

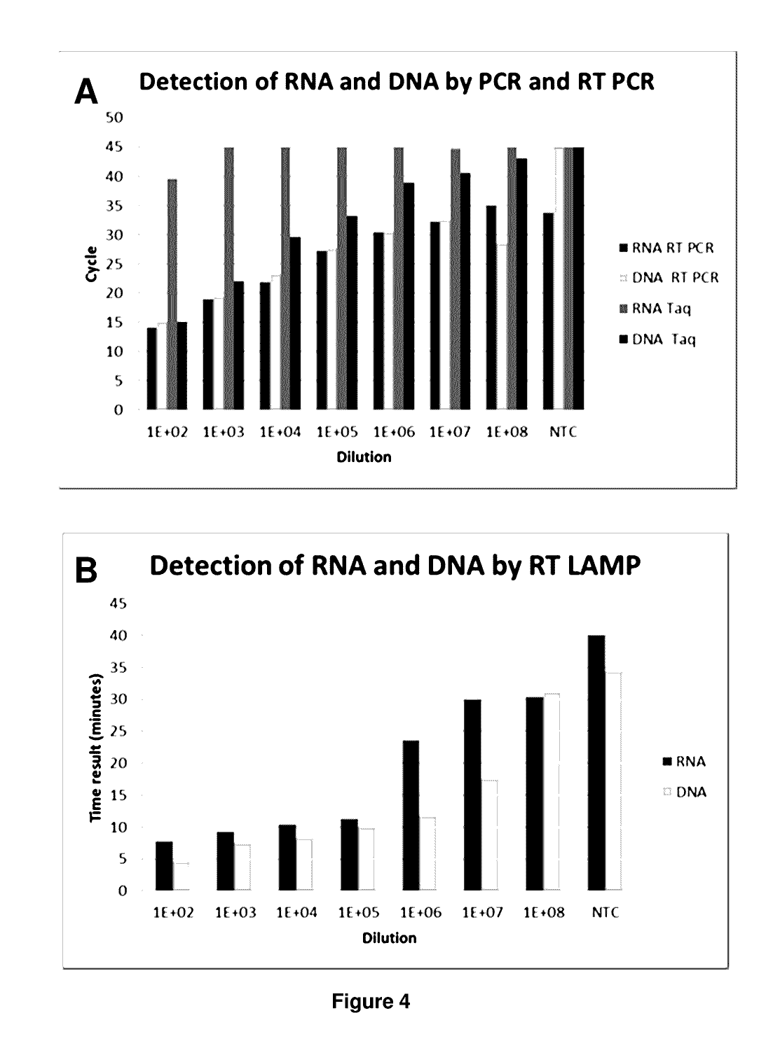

The relative efficiency of detecting extracted RNA and DNA by the RT LAMP method of this invention versus standard and reverse transcription PCR was tested to establish that the reaction detects rRNA rather than the gDNA gene that encodes it. A dilution series of the extracted RNA and DNA samples of Blastomyces dermatitidis were detected using standard PCR (EconoTaq, Lucigen) and RT PCR (Path-ID Multiplex One-Step RT-PCR Kit, Life Technologies) according to the respective manufacturer's recommendations. Results are shown in FIG. 4. Panel A. The same dilution series of RNA and DNA were tested for sensitivity of detection by the RT LAMP process of this invention using the conditions described above. These results are shown in FIG. 4, Panel B. The RT LAMP results are much more concordant with RT PCR detection than the standard PCR detection, providing strong evidence that the reaction directly detects the rRNA. Primer sets BL29 and BL55 (FIG. 3) had similar sensitivities (not shown). The BL29 primer set was used for the remaining work described.

Direct Detection of Fungal Cells by rRNA LAMP in the Absence and Presence of Sample Matrix

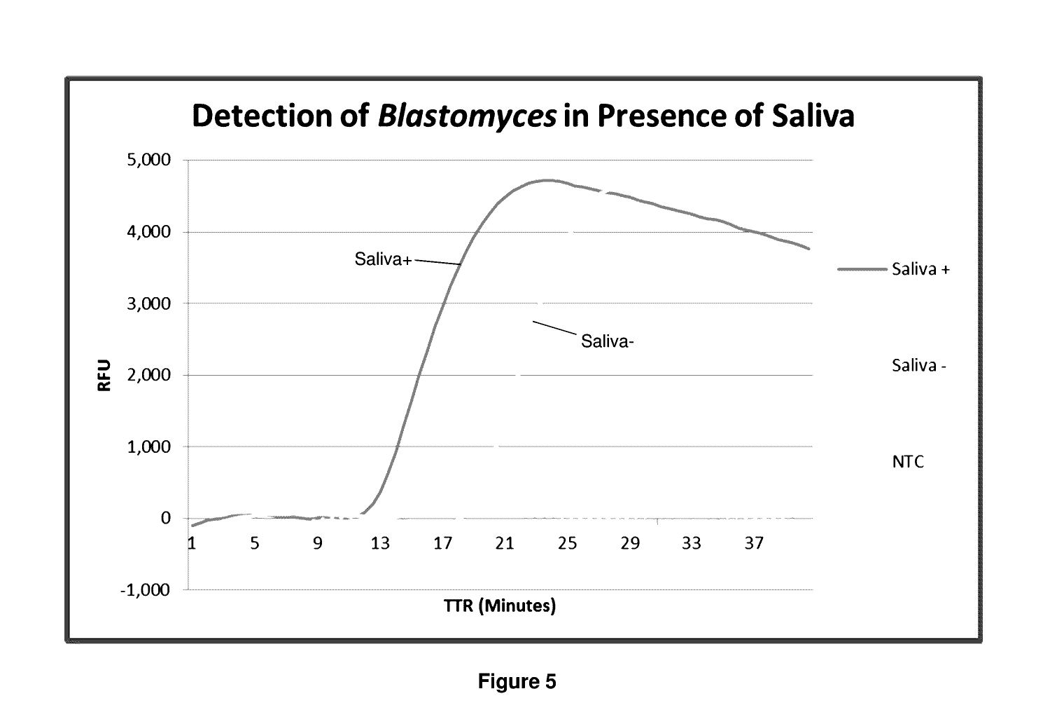

Blastomyces cells were grown in culture to circa 10⁶ cells per milliliter. Two microliters of culture was added to a final volume of 25 microliters and tested directly without additional processing using the materials and methods described in Example 3. Interference by saliva was tested by substituting saliva for water in the sample and testing sensitivity versus water resuspension (FIG. 5). The saliva did not inhibit the reaction and may have slightly shortened time to detection.

### Example 3

### Dry Formulation of Amplification Mix

### Summary

A dry, stable isothermal amplification mix that can be stored at room temperature for extended periods of time would be useful for detecting organisms in point-of-care settings and in the field. The present example demonstrates efficacy of a dry amplification mix.

### Methods and Results

B. dermatidtidis DNA was obtained. A 1.2 kb PCR amplicon covering the rRNA complex was generated. The 1.2 kb product size is consistent with single-copy amplification. An amplification mix was formulated and lyophilized so that reconstitution to 25 μl with B. dermatidtidis amplicon sample suspended in water containing 2 mM Fiona Green dye would result in the following concentrations: 0.2 μM primer BL29F3 (SEQ ID NO:8), 0.2 μM primer BL29B3 (SEQ ID NO:9), 1.6 μM primer BL29FIP (SEQ ID NO:10), 1.6 μM primer BL29BIP (SEQ ID NO: 1), 0.4 μM primer BL29LF1 (SEQ ID NO:12), 0.4 μM primer BL29LB1 (SEQ ID NO:13), 20 mM K-MOPS pH 7.9, 10 mM NH₄SO₄, 0.8 mM dATP, 0.8 mM dCTP, 0.8 mM dGTP, 0.8 mM dTTP, 10 mM MgSO₄. 0.3% CHAPS, 0.8% Trehalose, 4.5 U/μl PYROPHAGE 3173 exo-DNA Polymerase (Lucigen Corporation, Madison, Wis.). Limit of detection (LOD) using LAMP was determined. The analytical sensitivity to the single copy target was 40 copies in 22 minutes with no significant background detection. The dried amplification mix used in this example has stability at room temperature for over six months. Thus, the present example shows that a dried amplification mix containing polymerase enzyme can be used in the methods described herein.

### Example 4

### Direct Detection of Cellular rRNA with and without Enzyme Lysis or Cell Agitation

### Summary

The simplest possible sample preparation for point-of-care use is direct detection of cultured cells without pretreatment. The present example addresses the need for a rapid and easy method of extracting amplifiable nucleic acid from cells without pretreatment for point-of-case use.

### Methods and Results

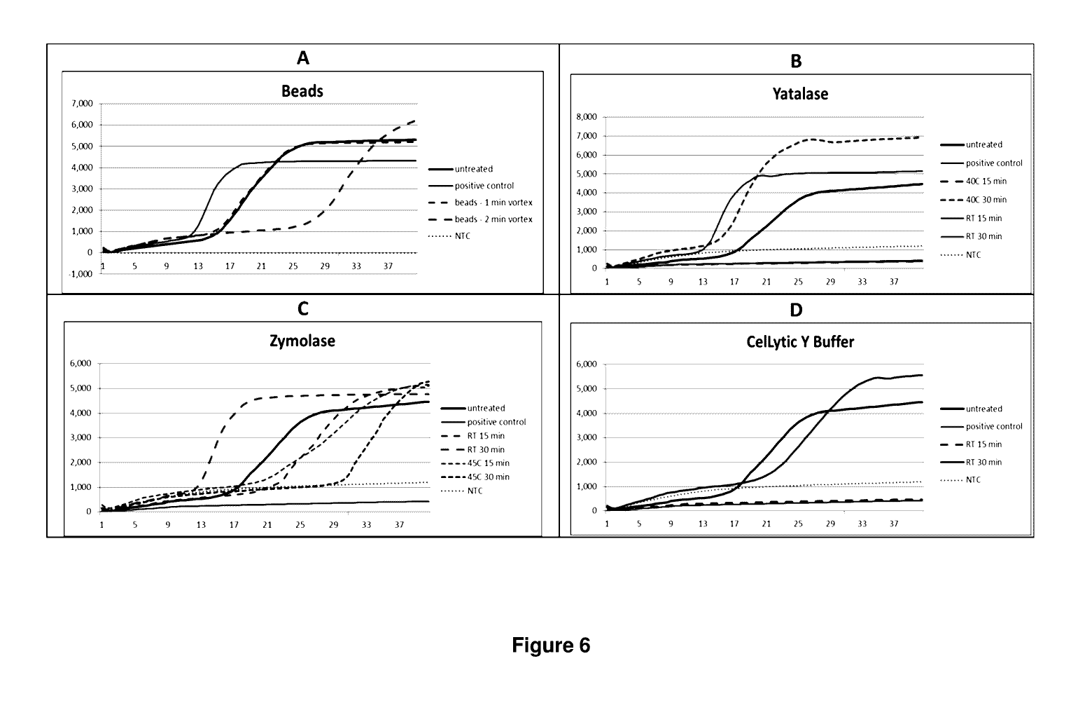

A culture of Blastomyces dermatitidis (ATCC 26199) was obtained, and a lyophilized amplification mix was formulated as in Example 3. Tris buffer (pH 7.5) was used to reconstitute the cells. The reconstituted cells were mixed with the lyophilized amplification mix, and RT LAMP was performed as outlined in Example 2. The results of direct detection without pretreatment (“untreated” shown in FIG. 6, Panels A, B, C and D) were suitable for the intended use in terms of sensitivity and time to result. Additional lysis treatments were tested to determine whether such treatments improved the assay. Agitation with silica beads for one and two minutes, as indicated, was used immediately prior to detection (FIG. 6. Panel A). Pretreatment with three enzymes commonly used for lysis of fungal cells, Yatalase (Clontech, Mountain View, Calif.). Zymolase (Zymo Research, Irvine, Calif.) and CellLytic Y (Sigma-Aldrich) was also performed according to the respective manufacturers' recommendations at varied temperatures as indicated (FIG. 8, Panels B, C, and D). Neither the enzymes nor the bead beating improved the performance of the assay enough to justify the time required for the pretreatment. It is hypothesized that an amplification mix containing detergents, and/or reducing agents, such as the amplification mixes described herein, destabilize cell walls and release rRNA for amplification following thermal and osmotic shock.

### Example 5

### Direct Detection of HPV E6/E7 mRNA

### Summary

To distinguish mRNA expressed from the E6/E7 gene of integrated HPV from its cognate gDNA sequence. LAMP primer sets were designed to span the splice site of the intron that is excised during gene expression. The common HeLa cell line contains an integrated HPV18 virus and constitutively expresses E6/E7 mRNA. The primers span an intron boundary, i.e. a splice site, making the spliced mRNA readily distinguishable from the corresponding gDNA.

### Methods and Results

The gDNA and the spliced mRNA sequences of HPV18 are represented by SEQ ID NO: 14 and SEQ ID NO:15, respectively. mRNA was extracted from HeLa cells and used as a target for LAMP detection. 0.2 μM primer HPV70F3 (SEQ ID NO:16), 0.2 μM primer HPV70B3 (SEQ ID NO:17), 1.6 μM primer HPV70FIP (SEQ ID NO:18), 1.6 μM primer HPV70BIP (SEQ ID NO:19), 0.4 μM primer HPV70LF2 (SEQ ID NO:20), 0.4 μM primer HPV70LB2 (SEQ ID NO:21), 20 mM K-MOPS pH 7.9, 10 mM NH₄SO₄0.0.8 mM dATP, 0.8 mM dCTP, 0.8 mM dGTP, 0.8 mM dTTP, 10 mM MgSO₄. 0.3% CHAPS, 2 mM Fiona Green dye, 0.72 U/μl PYROPHAGE 3173 exo-DNA Polymerase was used. 2.0 μl of each sample dilution was added to 23.0 μl of amplification mix and the reactions transferred immediately to a Bio-Rad cfx96 realtime PCR thermocyler run isothermally at 68° C. for the duration of the test. Fluorescence was monitored using the FAM/SYBR setting on the instrument at 30 second intervals. Time to result (TTR) was determined based on the time for the relative fluorescence to cross a theoretical threshold set at 10% of the maximum.

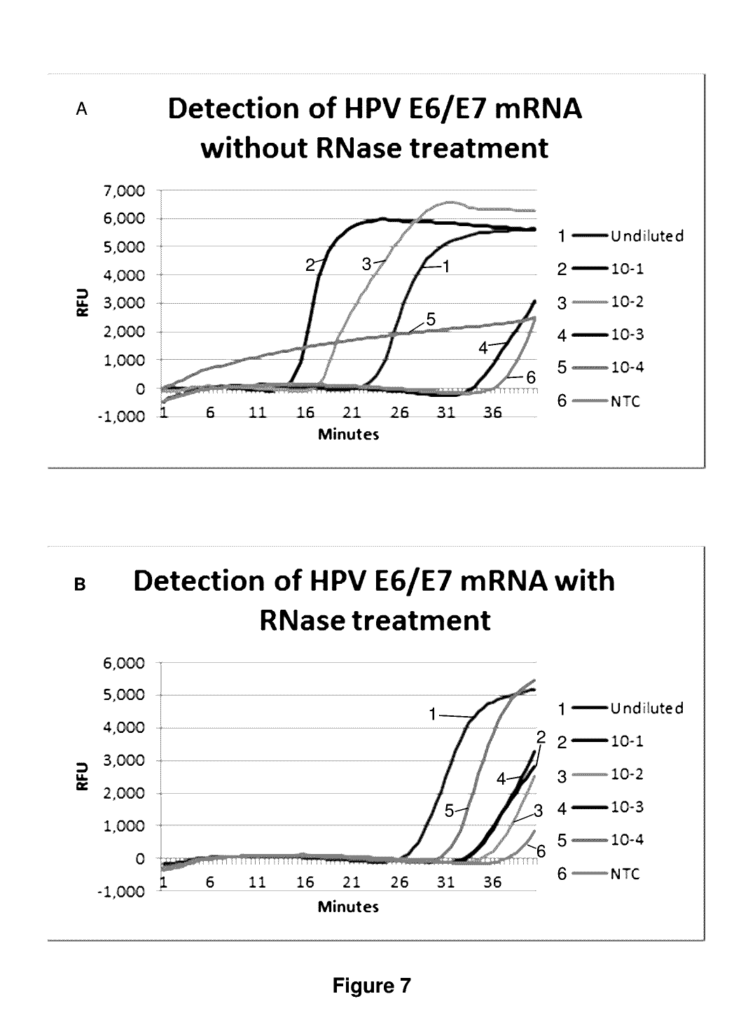

Results are shown in FIG. 7. To further establish the identity of the RNA target rather than gDNA, the extracted samples were divided into two aliquots. One aliquot was treated for 30 minutes at 37 degrees C. with 10 μg RNase and the other was not treated enzymatically. The detection times were compared for the RNase treated and untreated samples are shown in FIG. 7. The significant delay between the RNase treated sample compared to the untreated sample supports the detection of RNA rather than the alternative explanation that DNA was the detection target. We predict that the mRNA and other types of RNA is capable of being detected from whole-cell samples as described above in Examples 1 and 4 for rRNA. We also predict that the mRNA and other types of RNA is capable of being detected, with purified mRNA or whole cell-samples, from dried reagents as described above in Example 3 for rRNA.

### NON-PATENT REFERENCES

Amann R I, Binder B J, Olson R J. Chisholm S W, Devereux R, Stahl D A. Combination of 16S rRNA-targeted oligonucleotide probes with flow cytometry for analyzing mixed microbial populations. Applied and Environmental Microbiology. 1990; 56(6):1919-1925.
Boulet G A. Micalessi I M, Horvath C A, Benoy I H. Depuydt C E. Bogers J J. Nucleic acid sequence-based amplification assay for human papillomavirus mRNA detection and typing: evidence for DNA amplification. J Clin Microbiol. 2010 July; 48(7):2524-9.
Cole, J. R., B. Chai, T. L. Marsh, R. J. Farris. Q. Wang. S. A. Kulam, S. Chandra. D. M. McGarrell, T. M. Schmidt, G. M. Garrity, and J. M. Tiedje. 2003. The Ribosomal Database Project (RDP-II): previewing a new autoaligner that allows regular updates and the new prokaryotic taxonomy. Nucleic Acids Res. 31(1):442-443.
Cole, J. R., B. Chai, R. J. Farris, Q. Wang, S. A. Kulam. D. M. McGarrell, G. M. Garrity, and J. M. Tiedje. 2005. The Ribosomal Database Project (RDP-II): sequences and tools for high-throughput rRNA analysis. Nucleic Acids Res. 33 (Database Issue):D294-D296.
Cole, J. R., B. Chai, R. J. Farris. Q. Wang, A. S. Kulam-Syed-Mohideen. D. M. McGarrell, A. M. Bandela, E. Cardenas, G. M. Garrity, and J. M. Tiedje. 2007. The ribosomal database project (RDP-II): introducing myRDP space and quality controlled public data. Nucleic Acids Res. 35 (Database issue): D169-D172.
Cole. J. R., Q. Wang, E. Cardenas. J. Fish. B. Chai, R. J. Farris, A. S. Kulam-Syed-Mohideen, D. M. McGarrell, T. Marsh, G. M. Garrity, and J. M. Tiedje. 2009. The Ribosomal Database Project: improved alignments and new tools for rRNA analysis. Nucleic Acids Res. 37 (Database issue): D141-145.
Cole J. R., Q. Wang, B. Chai and J. M. Tiedje. 2011. The Ribosomal Database Project: sequences and software for high-throughput rRNA analysis. In F. J. de Bruijn (ed), Handbook of Molecular Microbial Ecology I: Metagenomics and Complementary Approaches. J. Wiley & Sons, Inc., Hoboken. N J.
Cole, J. R., Q. Wang. J. A. Fish. B. Chai, D. M. McGarrell, Y. Sun, C. T. Brown, A. Porras-Alfaro, C. R. Kuske, and J. M. Tiedje. 2014. Ribosomal Database Project: data and tools for high throughput rRNA analysis Nucl. Acids Res. 42 (Database issue):D633-D642.
C. L. Liu. H. W. Ai. W. P. Wang, L. Chen. H. B. Hu, T. Ye, X. H. Zhu, F. Wang, Y. L. Liao, Y. Wang. G. Ou. L. Xu. M. Sun, C. Jian, Z. J. Chen. L. Li, B. Zhang. L. Tian, B. Wang. S. Yan, Z. Y. Sun. Comparison of 16S rRNA gene PCR and blood culture for diagnosis of neonatal sepsis, Archives de Péliatrie, 2014, 21, 2, 162
Chetverin, Alexander B., Helena V. Chetverina, and Alexander V. Munishkin. “On the nature of spontaneous RNA synthesis by Qβ replicase.” Journal of molecular biology 222.1 (1991): 3-9.
Dark P M. Dean P, Warhurst G. Bench-to-bedside review: the promise of rapid infection diagnosis during sepsis using polymerase chain reaction-based pathogen detection. Crit Care. 2009:13(4):217.
Deiman, Birgit, Pierre van Aarle, and Peter Sillekens. “Characteristics and applications of nucleic acid sequence-based amplification (NASBA).” Molecular biotechnology 20.2 (2002): 163-179.
Dockter J. Schroder A. Hill C, Guzenski L, Monsonego J, Giachetti C. Clinical performance of the APTIMA HPV Assay for the detection of high-risk HPV and high-grade cervical lesions. J Clin Virol. 2009 July; 45 Suppl 1:S55-61.
Frickmann H, Loderstaedt U, Racz P, Tenner-Racz K, Eggert P, et al. (2015) Detection of tropical fungi in formalin-fixed, paraffin-embedded tissue: still an indication for microscopy in times of sequence-based diagnosis? Biomed Res Int 2015: 938721.
Fish. J. A., B. Chai, Q. Wang. Y. Sun, C. T. Brown, J. M. Tiedje, and J. R. Cole. 2013. FunGene: the Functional Gene Pipeline and Repository. Front. Microbiol. 4:291.
Edman, Carl F., et al. “Pathogen analysis and genetic predisposition testing using microelectronic arrays and isothermal amplification.” Journal of investigative medicine: the official publication of the American Federation for Clinical Research 48.2 (2000): 93-101.
G. N. Hartmeyer, U. S. Justesen, Direct 16S rRNA gene sequencing of polymicrobial culture-negative samples with analysis of mixed chromatograms, Journal of Medical Microbiology, 2010, 59, 4, 486
Gonzalez I L, Gorski J L. Campen T J. Dorney D J. Erickson J M, Sylvester J E. Schmickel R D. Variation among human 28S ribosomal RNA genes. Proc Natl Acad Sci U.SA. 1985 November; 82(22):7666-70.
Han. Eun-Taek. et al. “Detection of four Plasmodium species by genus- and species-specific loop-mediated isothermal amplification for clinical diagnosis.” Journal of clinical microbiology 45.8 (2007): 2521-2528.
Haanshuus C G, Mohn S C, Morch K. Langeland N. Blomberg B, et al. (2013) A novel, single-amplification PCR targeting mitochondrial genome highly sensitive and specific in diagnosing malaria among returned travelers in Bergen. Norway. Malar J 12: 26.
Hagiwara M, Sasaki H, Matsuo K, Honda M, Kawase M, Nakagawa H. Loop-mediated isothermal amplification method for detection of human papillomavirus type 6, 11, 16, and 18. J Med Virol. 2007 May:79(5):605-15.
Hirayama, Hiroki. et al. “Rapid sexing of water buffalo (Bubalus bubalis) embryos using loop-mediated isothermal amplification.” Theriogenology 66.5 (2006): 1249-1256.
Hofmann N, Mwingira F. Shekalaghe S, Robinson L J, Mueller I, Felger I. Ultra-sensitive detection of Plasmodium falciparum by amplification of multi-copy subtelomeric targets. PLoS Med. 2015 Mar. 3; 12(3):e1001788.
Huy N T, Hang le T T, Boamah D, Lan N T, Van Thanh P. et al. (2012) Development of a single-tube loop-mediated isothermal amplification assay for detection of four pathogens of bacterial meningitis. FEMS Microbiol Lett 337: 25-30.
Jeantet D. Schwarzmann F, Tromp J, Melchers W J, van der Wurff A A, Oosterlaken T. Jacobs M, Troesch A. NucliSENS EasyQ HPV v1 test—Testing for oncogenic activity of human papillomaviruses. J Clin Virol. 2009 July; 45 Suppl 1:S29-37.
Jenkins C. Ling C L, Ciesielczuk H L, Lockwood J, Hopkins S, et al. (2012) Detection and identification of bacteria in clinical samples by 16S rRNA gene sequencing: comparison of two different approaches in clinical practice. J Med Microbiol 61: 483-488.
Jonas, Vivan, et al. “Detection and identification of Mycobacterium tuberculosis directly from sputum sediments by amplification of rRNA.” Journal of clinical microbiology 31.9 (1993): 2410-2416.
Kievits, Tim, et al. “NASBA TM isothermal enzymatic in vitro nucleic acid amplification optimized for the diagnosis of HIV-1 infection.” Journal of virological methods 35.3 (1991): 273-286.
Kimura Y, de Hoon M J, Aoki S, Ishizu Y. Kawai Y, Kogo Y. Daub C O, Lezhava A. Arner E. Hayashizaki Y. Optimization of turn-back primers in isothermal amplification. Nucleic Acids Res. 2011 May; 39(9):e59.
Kox, L. F., et al. “PCR assay based on DNA coding for 16S rRNA for detection and identification of mycobacteria in clinical samples.” Journal of clinical microbiology 33.12 (1995): 3225-3233.
Larsen, N., G. J. Olsen, B. L. Maidak, M. J. McCaughey R. Overbeek. T. J. Macke, T. L. Marsh, and C. R. Woese. 1993. The ribosomal database project. Nucleic Acids Res. 21(13):3021-3023.
Liu Q. Lin X. Lin L, Yi L, Li H. Lin J M. A comparative study of three different nucleic acid amplification techniques combined with microchip electrophoresis for HPV16 E6/E7 mRNA detection. Analyst. 2015 Oct. 7; 140(19):6736-41.
Livingstone D M. Rohatensky M, Mintchev P, Nakoneshny S C, Demetrick D J, van Marle G, Dort J C. Loop mediated isothermal amplification (LAMP) for the detection and subtyping of human papillomaviruses (HPV) in oropharyngeal squamous cell carcinoma (OPSCC). J Clin Virol. 2016 February; 75:37-41.
Lo E. Yewhalaw D. Zhong D. Zemene E. Degefa T, et al. (2015) Molecular epidemiology of Plasmodium vivax and Plasmodium falciparum malaria among Duffy-positive and Duffy-negative populations in Ethiopia. Malar J 14: 84.
Luo L, Nie K, Yang M J. Wang M, Li J, Zhang C, Liu H T, Ma X J. Visual detection of high-risk human papillomavirus genotypes 16, 18, 45, 52, and 58 by loop-mediated isothermal amplification with hydroxynaphthol blue dye. J Clin Microbiol. 2011 October; 49(10):3545-50.
Maidak. B. L., N. Larsen. M. J. McCaughey, R. Overbeek. G. J. Olsen. K. Fogel, J. Blandy, and C. R. Woese. 1994. The Ribosomal Database Project. Nucleic Acids Res. 22(17):3485-3487.
Maidak, B. L., N. Larsen, M. J. McCaughey. R. Overbeek, G. J. Olsen, K. Fogel. J. Blandy, and C. R. Woese. 1996. The Ribosomal Database Project (RDP). Nucleic Acids Res. 24(1):82-85.
Maidak, B. L., G. J. Olsen. N. Larsen. R. Overbeek, M. J. McCaughey, and C. R. Woese. 1997. The RDP (Ribosomal Database Project). Nucleic Acids Res. 25(1):109-111.
Maidak, B. L., G. J. Olsen, N. Larsen, R. Overbeek. M. J. McCaughey, and C. R. Woese. 1999. A new version of the RDP (Ribosomal Database Project). Nucleic Acids Res. 27(1):171-173.
Maidak, B. L., J. R. Cole, C. T. Parker, Jr., G. M. Garrity, N. Larsen. B. Li. T. G. Lilburn, M. J. McCaughey, G. J. Olsen. R. Overbeek, S. Pramanik, T. M. Schmidt, J. M. Tiedje, and C. R. Woese. 2000. The RDP (Ribosomal Database Project) continues. Nucleic Acids Res. 28(1):173-174.
Maidak, B. L., J. R. Cole, T. G. Lilburn. C. T. Parker. Jr., P. R. Saxman, R. J. Farris, G. M. Garrity, G. J. Olsen, T. M. Schmidt, and J. M. Tiedje. 2001. The RDP-II (Ribosomal Database Project). Nucleic Acids Res. 29(1): 173-174.
Mori, Yasuyoshi, et al. “Detection of loop-mediated isothermal amplification reaction by turbidity derived from magnesium pyrophosphate formation.” Biochemical and biophysical research communications 289.1 (2001): 150-154.
Nickerson, Deborah A., et al. “Automated DNA diagnostics using an ELISA-based oligonucleotide ligation assay.” Proceedings of the National Academy of Sciences 87.22 (1990): 8923-8927.
Notomi, Tsugunori, et al. “Loop-mediated isothermal amplification of DNA.” Nucleic acids research 28.12 (2000): e63-e6
Olsen, G. J., R. Overbeek, N. Larsen, T. L. Marsh, M. J. McCaughey, M. A. Maciukenas, W. M. Kuan, T. J. Macke, Y. Xing, and C. R. Woese. 1992. The Ribosomal Database Project. Nucleic Acids Res. 20 Suppl:2199-2200.
Oriero C E, van Geertruyden J P. Jacobs J. D'Alessandro U, Nwakanma D. Validation of an apicoplast genome target for the detection of Plasmodium species using polymerase chain reaction and loop mediated isothermal amplification. Clin Microbiol Infect. 2015 Mar. 6. pii: S1198-743X(15)00317-1.
Ptaszynska A A, Borsuk G, Wozniakowski G, Gnat S. Malek W (2014) Loop-mediated isothermal amplification (LAMP) assays for rapid detection and differentiation of Nosema apis and N. ceranae in honeybees. FEMS Microbiol Lett 357: 40-48.
Ratnam S. Coutlee F. Fontaine D, et al. Clinical Performance of the PreTect HPV-Proofer E6/E7 mRNA Assay in Comparison with That of the Hybrid Capture 2 Test for Identification of Women at Risk of Cervical Cancer. Journal of Clinical Microbiology. 2010; 48(8):2779-2785.
Ravindran A, Levy J. Pierson E, Gross D C (2015) Loop-Mediated Isothermal Amplification Procedure (LAMP) for Detection of the Potato Zebra Chip Pathogen “Candidatus Liberibacter solanacearum”. Methods Mol Biol 1302: 85-97.
Sotiriadou. Isaia, and Panagiotis Karanis. “Evaluation of loop-mediated isothermal amplification for detection of Toxoplasma gondii in water samples and comparative findings by polymerase chain reaction and immunofluorescence test (IFT).” Diagnostic microbiology and infectious disease 62.4 (2008): 357-365.
Stary. Angelika. et al. “Performance of transcription-mediated amplification and Ligase chain reaction assays for detection of chlamydial infection in urogenital samples obtained by invasive and noninvasive methods.” Journal of clinical microbiology 36.9 (1998): 2666-2670.
Steenkeste N, Incardona S, Chy S. Duval L, Ekala M T, et al. (2009) Towards high-throughput molecular detection of Plasmodium: new approaches and molecular markers. Malar J 8: 86.
Stender, Henrik, et al. “PNA for rapid microbiology.” Journal of Microbiological Methods 48.1 (2002): 1-17.
Tomita, Norihiro, et al. “Loop-mediated isothermal amplification (LAMP) of gene sequences and simple visual detection of products.” Nature protocols 3.5 (2008): 877-882.
Walker. G. Terrance, et al. “Isothermal in vitro amplification of DNA by a restriction enzyme/DNA polymerase system.” Proceedings of the National Academy of Sciences 89.1 (1992): 392-396.
Wang, Q. G. M. Garrity, J. M. Tiedje, and J. R. Cole. 2007. Naïve Bayesian Classifier for Rapid Assignment of rRNA Sequences into the New Bacterial Taxonomy. Appl Environ Microbiol. 73(16):5261-5267.
Wang, Q., J. F. Quensen III, J. A. Fish, T.-K. Lee. Y. Sun, J. M. Tiedje, J. R. Cole. 2013. Ecological patterns of nifH genes in four terrestrial climatic zones explored with targeted metagenomics using FrameBot, a new informatics tool. mBio 4:e00592-13.
Xu, J., Millar, B. C., Moore. J. E., Murphy, K., Webb, H., Fox, A. J., Cafferkey, M. and Crowe, M. J. 2003. Employment of broad-range 16S rRNA PCR to detect aetiological agents of infection from clinical specimens in patients with acute meningitis—rapid separation of 16S rRNA PCR amplicons without the need for cloning. Journal of Applied Microbiology, 94: 197-206.
Xu Z. Li L. Chu J, Peters B M, Harris M L. et al. (2012) Development and application of loop-mediated isothermal amplification assays on rapid detection of various types of staphylococci strains. Food Res Int 47: 166-173.
Xu G. Hu L, Zhong H, Wang H, Yusa S. Weiss T C, Romaniuk P J, Pickerill S. You Q. 2012. Cross priming amplification: mechanism and optimization for isothermal DNA amplification. Sci Rep. 2:246.

### PATENT REFERENCES

Battrell, C. Frederick, et al. “Lysing fluid sample on disposable microfluidic card, capturing separated components on solid substrate, removing interfering nucleic acids with buffer wash, amplifying and pumping over lateral flow strip for detection.” U.S. Pat. No. 7,416,892. 26 Aug. 2008.
Becker. Michael, and Kristin Livezey. “Methods, compositions and kits for isothermal amplification of nucleic acids.” U.S. patent application Ser. No. 11/517,117.
Brow, Mary Ann D., Victor I. Lyamichev, and David Michael Olive. “Rapid detection and identification of pathogens.” U.S. Pat. No. 6,372,424. 16 Apr. 2002.
Bruce, Ian J. “Nucleic acid amplification mediated microbial identification.” Science progress 77 (1992): 183-206.
Burg, J., et al. “Using specific oligonucleotide primers to enrich isothermal amplification reaction products with preferential nucleotide sequences; DNA amplification.” U.S. patent application Ser. No. 09/901,181.
Catanzariti, Luigi, et al. “Nucleic acid assays.” U.S. Pat. No. 6,528,632. 4 Mar. 2003.
Choi. Ko-bong. Joo-Won Rhee, and Sea-hee Kim. “Method of amplifying dna from ma in sample and use thereof.” U.S. patent application Ser. No. 13/715,955.
Church. George M., and Kun Zhang. “Amplification of trace amounts of nucleic acids.” U.S. Pat. No. 7,387,876. 17 Jun. 2008.
Dattagupta, Nanibhushan, et al. “Accumulating target nucleotide sequences; mixing primers, nucleotide sequences and preferential polymerase, heat, allow hybridization of primers and accumulation of nucleotide sequences, recover preferential sequences.” U.S. Pat. No. 6,214,587. 10 Apr. 2001.
Dattagupta, Nanibhushan, et al. “Accumulation of preferential nucleotide sequences; incubation of nucleotide sequences with polymerase, nucleotide triphosphates and primers, heat, allow hybridization, extend chains, accumulate target sequences.” U.S. Pat. No. 6,087,133. 11 Jul. 2000.
Dattagupta, Nanibhushan, et al. “Isothermal strand displacement nucleic acid amplification.” U.S. Pat. No. RE39,007. 7 Mar. 2006.
Davey, Cheryl, and Lawrence T. Malek. “Kit for simple and efficient isothermal accumulation of preferential nucleotide sequences.” U.S. Pat. No. 6,063,603. 16 May 2000.
Davey, Cheryl, and Lawrence T. Malek. “Nucleic acid amplification process.” U.S. Pat. No. 5,554,517. 10 Sep. 1996.
Davey, Cheryl, and Lawrence T. Malek. “Using Dna, Rna templates, primers and enzymes.” U.S. Pat. No. 5,409,818. 25 Apr. 1995.
Ecker, David J., et al. “Methods for rapid identification of pathogens in humans and animals.” U.S. Pat. No. 8,822,156. 2 Sep. 2014.
Everett, Karin D E, and Arthur A. Andersen. “Intergenic spacer target sequence for detecting and distinguishing Chlamydial species or strains.” U.S. Pat. No. 6,261,769. 17 Jul. 2001.
Fraiser. Melinda S., et al. “Strand displacement amplification using thermophilic enzymes.” U.S. Pat. No. 5,648,211. 15 Jul. 1997.
Gelfand, David H., and Thomas W. Myers. “Reverse transcription with Thermus thermophilus polymerase.” U.S. Pat. No. 5,407,800. 18 Apr. 1995.
Gu, Trent, Fen Huang, and James Robert Hartnett. “Bacillus stearothermophilus reverse transcription compositions and kits.” U.S. Pat. No. 7,094,539. 22 Aug. 2006.
Gu, Trent, Fen Huang, and James Robert Hartnett. “Method of reverse transcription.” U.S. Pat. No. 7,504,220. 17 Mar. 2009.
Himmler, Gottfried, and Thomas Schlederer. “Method for one step isothermal non-transcription based amplification of nucleic acids.” U.S. Pat. No. 6,033,881. 7 Mar. 2000.
Hogan. James John. et al. “hybridization assay using an oligonucleotide probe complementary to hybridize to a region of rRNA selected to be unique to a non-viral organism by comparing one or more variable region rRNA sequences with rRNA sequences of the organism to be detected; can distinguish between target and non-target species.” U.S. Pat. No. 7,090,972. 15 Aug. 2006.
Hogan, James John, et al. “Making a probe for use in nucleic acid hybridization assay by aligning ribosomal ma or encoding dna of target bacterial species to a nontarget species to identify a variable region, then designing target complementary sequence.” U.S. Pat. No. 6,150,517. 21 Nov. 2000.
Hogan. James John, et al. “Nucleic acid probes and methods for detecting chlamydia trachomatis.” U.S. Pat. No. 5,693,468. 2 Dec. 1997.
Hogan, James John. et al. “Oligonucleotide probes for the detection and/or quantitation of non-viral organisms.” U.S. Pat. No. 7,087,742. 8 Aug. 2006.
Hogan, James John, Jo Ann Kop, and Sherrol Hoffa McDonough. “Oligonucleotide able to hybridize to Neisseria gonorrhoeae nucleic acid r RNA but not Neisseria meningitis; probes for Mycobacterium, Legionella, Salmonella, Chlamydia, Campylobacter, Proteus, Enterococcus, Enterobacter, Escherichia, and/or Pseudomonas.” U.S. Pat. No. 7,172,863. 6 Feb. 2007.
Hogan. James John. “Nucleic acid probes and methods for detecting Escherichia coli.” U.S. Pat. No. 5,693,469. 2 Dec. 1997.
Inácio, Joāo, Orfeu Flores, and Isabel Spencer-Martins. “Efficient identification of clinically relevant yeasts combining a panfungal loop-mediated isothermal DNA amplification (LAMP) with hybridization to species-specific oligonucleotide probes.” Journal of Clinical Microbiology (2007).
Kacian, Daniel L., and Timothy J. Fultz. “Nucleic acid sequence amplification methods.” U.S. Pat. No. 5,399,491. 21 Mar. 1995.
Kohne, David E. “Ribosomal nucleic acid probes for detecting organisms or groups of organisms.” U.S. Pat. No. 5,723,597. 3 Mar. 1998.
Kramer. Fred R., Eleanor A. Miele, and Donald R. Mills. “Autocatalytic replication of recombinant RNA.” U.S. Pat. No. 4,786,600. 22 Nov. 1988.
Kurn, Nurith. “Methods and compositions for linear isothermal amplification of polynucleotide sequences, using a RNA-DNA composite primer.” U.S. Pat. No. 6,251,639. 26 Jun. 2001.
Lader, Eric S., Marianna Goldrick, and Matthew Winkler. “Controlling amplification of at least two target nucleotide sequences during primer dependent polymerase mediated deoxyribonucleic acid synthesis; preparing reaction mixture, incubation with primers, allowing synthesis.” U.S. Pat. No. 6,057,134. 2 May 2000.
Lampson, Bert C., and Jashree Velore. “RNA-dependent DNA polymerase from Geobacillus stearothermophilus.” U.S. Pat. No. 7,670,807. 2 Mar. 2010.
Lee, Helen Hwai-an, Magda Anastassova Dineva, and Fiona Frances Sarah Fletcher-Brown. “Nucleic acid amplification and testing.” U.S. patent application Ser. No. 12/524,261.
Löffert, Dirk, Andreas Missel, and Jie Kang. “Thermostable chimeric nucleic acid polymerases and uses thereof.” U.S. Pat. No. 8,637,288. 28 Jan. 2014.
Malek, Lawrence T., et al. “Enhanced nucleic acid amplification process.” U.S. Pat. No. 5,130,238. 14 Jul. 1992.
Marshall, Ronald L., John J. Carrino, and Joann C. Sustachek. “Amplification of RNA sequences using the ligase chain reaction.” U.S. Pat. No. 5,686,272. 11 Nov. 1997.
Masuda, Noriyoshi, et al. “Method of detecting acid-fast bacteria using ribosomal RNA as target.” U.S. Pat. No. 7,090,980. 15 Aug. 2006.
Masuda, Noriyoshi. et al. “Using 16s ribosomal RNA subregions for use as differentiative tools in taxonomic classification of mycobacterium.” U.S. patent application Ser. No. 10/737,894.
Millar, Douglas Spencer, John R. Melki, and Geoffrey W. Grigg. “Isothermal strand displacement amplification using primers containing a non-regular base.” U.S. Pat. No. 8,431,347. 30 Apr. 2013.
Mullis, Kary B., et al. “One of the first Polymerase Chain Reaction (PCR) patents.” U.S. Pat. No. 4,683,195. 28 Jul. 1987.
Murakami, Taku. “Primer generation rolling circle amplification.” U.S. patent application Ser. No. 11/813,142.
Rhee, Joo-Won, Sea-hee Kim, and Ko-bong Choi. “Method of amplifying target nucleic acid, method of analyzing target nucleic acid, kit for amplifying target nucleic acid, and composition for amplifying target nucleic acid.” U.S. patent application Ser. No. 14/082,250.
Segawa, Masaya, Motohiro Kondo, and Yutaka Takarada. “Reagent for nucleic acid amplification and process for nucleic acid amplification.” U.S. Pat. No. 6,261,773. 17 Jul. 2001.
Shapero, Michael. “Isothermal locus specific amplification.” U.S. patent application Ser. No. 11/186,120.
Shigemori, Yasushi, Takehiko Shibata, and Tsutomu Mikawa. “Extreme thermophile single-stranded DNA binding mutant protein, and nucleic acid isothermal amplification method of use thereof.” U.S. Pat. No. 7,973,131. 5 Jul. 2011.
Silverbrook, Kia, Mehdi Azimi, and Geoffrey Richard Facer. “Genetic analysis loc for nucleic acid amplification using an isothermal reaction.” U.S. patent application Ser. No. 13/150,054.
Spears, Patricia Anne, et al. “Oligonucleotide primer for strand displacement amplification of nucleotide sequences; for diagnosis, detection and amplification of microorganismal nucleotide sequences.” U.S. Pat. No. 6,156,508. 5 Dec. 2000.
Tanner, Nathan, Yinhua Zhang, and Thomas C. Evans. “Detection of Amplification Products.” U.S. patent application Ser. No. 13/834,919.
Todd, Alison V., Caroline J. Fuery, and Murray J. Cairns. “Detecting and quantitating target nucleic acid molecules in a sample via nucleic acid amplification.” U.S. Pat. No. 6,140,055. 31 Oct. 2000.
Van Gemen, Bob, Tim Kievits, and Peter F. Lens. “Quantification of nucleic acid.” U.S. Pat. No. 5,834,255. 10 Nov. 1998.
Walder, Joseph A., and Roxanne Y. Walder. “Nucleic acid hybridization and amplification method for detection of specific sequences in which a complementary labeled nucleic acid probe is cleaved.” U.S. Pat. No. 5,403,711. 4 Apr. 1995.
Wang, Sha-Sha, and David Wolfe. “Amplification and detection of organisms of the Chlamydiaceae family.” U.S. Pat. No. 6,682,889. 27 Jan. 2004.
Webster Jr, John A. “Method for detecting prokaryotic organisms.” U.S. Pat. No. 5,348,854. 20 Sep. 1994.
Wharam, Susan D., et al. “Specific detection of DNA and RNA targets using a novel isothermal nucleic acid amplification assay based on the formation of a three-way junction structure.” Nucleic acids research 29.11 (2001): e54-e54.
Zhou, Jizhong, and Liyou Wu. “using an isothermal DNA polymerase to randomly and representatively amplify genomic DNA of the microorganisms, hybridizing it to a polynucleotide microarray that can differentiate genes, species, or strains of microorganisms and measuring hybridization signals on the microarray to quantify the microbes.” U.S. Pat. No. 7,759,057. 20 Jul. 2010.

What is claimed is:

1. A method of detecting RNA comprising:
heating a cell comprising RNA in a solution to release the RNA from the cell;
reverse transcribing the RNA into DNA with an enzyme at a reverse-transcription temperature;
amplifying the DNA with the enzyme at an amplification temperature; and
detecting the amplified DNA, wherein:
the enzyme is thermostable at the reverse-transcription temperature, is thermostable at the amplification temperature, has reverse transcriptase activity, has DNA-dependent DNA polymerase activity, and has strand displacement activity; and
the reverse-transcription temperature and the amplification temperature are substantially the same.

2. The method of claim 1 wherein the reverse-transcription temperature and the amplification temperature are each within 15° C. of each other.

3. The method of claim 1 wherein the reverse-transcription temperature and the amplification temperature are each within a range of from 65° C. to 90° C.

4. The method of claim 1 comprising maintaining a substantially constant temperature during the reverse transcribing, from the reverse transcribing to the amplifying, and during the amplifying.

5. The method of claim 4 wherein the substantially constant temperature is within a range spanning 15° C.

6. The method of claim 1 wherein the heating comprises heating the cell to a temperature substantially the same as the reverse-transcription temperature and the amplification temperature.

7. The method of claim 1 comprising maintaining a substantially constant temperature during the heating, from the heating to the reverse transcribing, during the reverse transcribing, from the reverse transcribing to the amplifying, and during the amplifying.

8. The method of claim 7 wherein the substantially constant temperature is within a range spanning 15° C.

9. The method claim 1 wherein the heating, the reverse transcribing, and the amplifying are performed without adding additional reagents during or between these steps.

10. The method of claim 1 wherein the heating, the reverse transcribing, the amplifying, and the detecting are performed without adding additional reagents during or between these steps.

11. The method of claim 1 wherein the heating, the reverse transcribing, the amplifying, and the detecting are performed in a single container at a substantially constant temperature without adding additional reagents during or between these steps.

12. The method of claim 11 wherein the substantially constant temperature is within a range spanning 15° C.

13. The method of claim 1 wherein the RNA is ribosomal RNA (rRNA).

14. The method of claim 1 wherein the RNA is messenger RNA (mRNA).

15. The method of claim 1 wherein the solution comprises a primer that is complimentary to a ribosomal RNA sequence that is conserved among cells of a first cell type and is not present in ribosomal RNA of cells of a second cell type.

16. The method of claim 1 wherein the amplifying comprises loop-mediated isothermal amplification.

17. The method of claim 1 wherein the solution comprises a bodily fluid.

18. The method of claim 1 further comprising mixing a liquid with dried solution components to generate the solution, wherein the dried solution components comprise the enzyme.

19. The method of claim 18, wherein the liquid comprises a bodily fluid.

20. The method of claim 1 further comprising mixing a liquid comprising the cell with dried solution components to generate the solution, wherein the dried solution components comprise the enzyme.

21. The method of claim 1 wherein the heating does not include enzymatic digestion or physical agitation of the cell and wherein the heating releases the RNA from the cell without enzymatic digestion or physical agitation of the cell.
